(19) United States
(12) Patent Application Publication
(10) Pub. No.: US 2023/0173037 A1
(43) Pub. Date: Jun. 8, 2023

(54) Manipulating Glycogen in Alzheimer’s Disease, Epilepsy, Traumatic Brain Injury, and ALS as a Treatment

(71) Applicant: University of Kentucky Research Foundation, Lexington, KY (US)

(72) Inventors: Matthew S. Gentry, Lexington, KY (US); Ramon C. Sun, Lexington, KY (US); Lance A. Johnson, Lexington, KY (US); Peter T. Nelson, Lexington, KY (US); Frank Gilliam, Lexington, KY (US)

(21) Appl. No.: 17/908,827

(22) PCT Filed: Mar. 9, 2021
(86) PCT No.: PCT/US2021/021562

§ 371 Date: Sep. 1, 2022

Related U.S. Application Data
(60) Provisional application No. 62/987,208, filed on Mar. 9, 2020.

(51) Int. Cl. A61K 38/47; C12N 9/26; C12N 15/113; C07K 16/18; A61K 31/366; A61K 31/085; A61K 31/4436; A61K 31/438; A61K 31/4196; A61P 25/28

(52) U.S. Cl. CPC A61K 38/47; A61K 31/085; A61K 31/366; A61K 31/438; A61K 31/4196; A61K 31/4436; A61P 25/28; C07K 16/18; C12N 9/2414; C12N 15/1137; C12Y 302/01001; C07K 2319/00; C12N 2310/11; C12N 2310/531

(57) ABSTRACT

A method for treating a neurodegenerative disease is provided. The method for treating a neurodegenerative disease includes administering, to a subject in need thereof, one or more of a small molecule glycogen synthase (GYS) inhibitor, an antisense oligonucleotide targeting glycogen synthase, an antibody-enzyme fusion compound targeting polyglucosan bodies (PGBs), or combinations thereof.

### RELATED APPLICATIONS

This application claims the benefit of U.S. Provisional Application Serial No. 62/987,208, filed Mar. 9, 2020, the entire disclosure of which is incorporated herein by this reference.

### GOVERNMENT INTEREST

This invention was made with government support under grant number R01 NS070899-06-AD-s awarded by the National Institute of Neurological Disorders and Stroke (NINDS), National Institutes of Health (NIH). The government has certain rights in the invention.

### SEQUENCE LISTING

The instant application contains a Sequence Listing which has been submitted in ASCII format via EFS-Web and is hereby incorporated by reference in its entirety. The ASCII copy of the Sequence Listing, which was created on Mar. 9, 2021, is named 13177N-2366WO.txt and is 41.3 kilobytes in size.

### TECHNICAL FIELD

The present disclosure is directed to compounds and methods for treating neurodegenerative diseases by targeting glycogen aggregates. In particular, the disclosure is directed to compounds and methods for treating neurodegenerative diseases by preventing and/or clearing glycogen formation in the brain.

### BACKGROUND

Glycogen (1-5 µm in diameter) is a branched polymer of glucose synthesized by glycogen synthase (GYS), forming α-1,4-glycosidic linkages, and branching enzyme (BE), adding a-1,6-glycosidic linkages every 10-15 glucose residues (FIG. 1). Additionally, phosphate is covalently attached at glucose hydroxyls during synthesis, the removal of which requires the glycogen phosphatase laforin. Glycogen architecture describes the frequency of branches, glucose chain length, total phosphate esters, and phosphate position (FIG. 1). These architectural properties define the granular size, crystallinity, and solubility of a glycogen molecule.

Glycogen serves as the primary mammalian storage carbohydrate. However, the function of brain glycogen extends beyond a simple energy reserve, and includes a central role in maintaining brain homeostasis (FIG. 2). Glycogen is primarily found in astrocytes, and glycogen-derived glucose from astrocytes supports neuronal function by contributing to the lactate-pyruvate shuttle to maintain glutamatergic synaptic activity and mitochondrial bioenergetics. Furthermore, a small molecule inhibitor that prevents glucose release from glycogen leads directly to aberrant metabolism in astrocytes and subsequent neurotoxicity in preclinical models. Genetically engineered mice that cannot form glycogen in the brain have defects in short-term memory consolidation, long-term memory formation, and cannot maintain long-term potentiation of synaptic strength. Together, these data support the notion that glycogen serves an integral role in maintaining cerebral function (FIG. 2).

Aberrant glycogen metabolism leads to the accumulation of large (50-100 µm), hyperbranched and hyperphosphorylated glycogen aggregates called polyglucosan bodies (PGBs). PGBs are water-insoluble aggregates that are no longer efficiently degraded. On a molecular level, PGB accumulation results in increased ER stress, autophagy impairment, and epigenetic alterations. Electron microscopy from the 1980s demonstrated the accumulation of PGBs in the aging brain. Large, densely packed glycogen granules were observed in human postmortem specimens aged 60 or older, but not present in the younger control group aged 26-35. Since then, age-associated changes in glycogen metabolism and PGB accumulations have been demonstrated in Drosophila, mice, and human. Furthermore, an inducible knockout of Gys in Drosophila ― which ablates glycogen synthesis — restored locomotor decline with age and also increased lifespan.

The onset of Alzheimer’s disease (AD) increases in incidence with age, and an increasing aging demographic means the number of individuals suffering from AD is expanding rapidly. AD patients suffer from a slow and inevitable decline in memory and cognitive function through neurodegeneration. Changes in brain metabolism are hallmarks of both aging and AD. Glucose is the primary substrate for brain metabolism, and its availability and proper utilization are critical for synaptic communication and normal brain homeostasis. Brain glucose hypometabolism, evidenced by a decrease in the uptake of ¹⁸F-deoxyglucose (FDG) as measured by PET imaging, is now considered one of the hallmarks of AD. Clinical AD symptoms are intimately linked with glucose hypometabolism, and the degree of glucose hypometabolism strongly correlates with the severity of clinical symptoms. Interestingly, brain glucose hypometabolism is especially prominent in several groups with increased risk of AD, including carriers of the E4 allele of APOE. APOE4 is currently the most significant genetic risk factor for late onset AD. To this end, the mechanism(s) that drives glucose hypometabolism remains a critical knowledge gap in AD research, and thus a barrier in designing novel therapeutic options.

Accordingly, while a number of amyloid-targeting therapies remain under investigation, there remains a need for new therapeutic targets and approaches for treating neurodegenerative diseases.

### SUMMARY

The presently-disclosed subject matter meets some or all of the above-identified needs, as will become evident to those of ordinary skill in the art after a study of information provided in this document.

This summary describes several embodiments of the presently-disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this summary or not. To avoid excessive repetition, this summary does not list or suggest all possible combinations of such features.

In some embodiments, the presently-disclosed subject matter includes a method for treating a neurodegenerative disease, the method including administering, to a subject in need thereof, one or more of a small molecule glycogen synthase (GYS) inhibitor, an antisense oligonucleotide targeting glycogen synthase, an antibody-enzyme fusion compound targeting polyglucosan bodies (PGBs), or combinations thereof. In some embodiments, the small molecule GYS inhibitor includes:

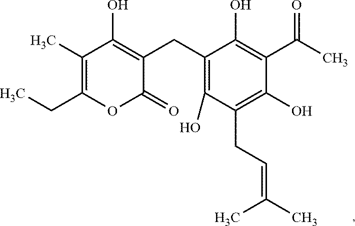

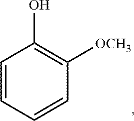

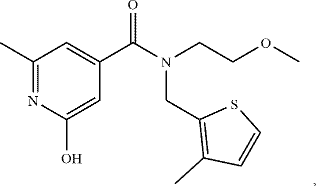

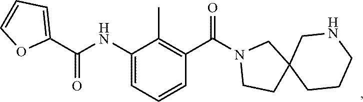

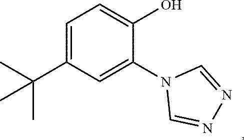

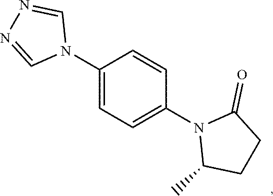

and a combination thereof. In some embodiments, the antisense oligonucleotide comprises a nucleotide sequence complementary to an mRNA sequence coding for brain-expressed GYS. In some embodiments, the antibody-enzyme fusion compound includes a cell-penetrating antibody comprising 3E10 linked to amylase or a variant thereof. In some embodiments, the antibody-enzyme fusion compound includes VAL-0417, VAL-1221, and combinations thereof.

In some embodiments, the method includes clearing PGB-like granules in the brain. In some embodiments, the method comprises clearing PGB-like granules from cytoplasm in the brain. In some embodiments, the one or more compounds comprise at least the antibody-enzyme fusion compound. In some embodimenst, the antibody-enzyme fusion compound comprises VAL-0417. In some embodiments, the method comprises inhibiting PGB formation in the brain. In some embodiments, the method comprises inhibiting PGB formation in cytoplasm in the brain. In some embodiments, the one or more compounds comprise at least one of the small molecule GYS inhibitor and the antisense oligonucleotide.

In some embodiments, the method includes first administering the antibody-enzyme fusion compound to clear existing PGB-like granules in the brain; and then administering at least one of the small molecule inhibitor and the antisense oligonucleotide to inhibit new PGB formation in the brain. In some embodiments, the neurodegenerative disease is selected from the group consisting of traumatic brain injury (TBI), Alzheimer’s Disease (AD), amyotrophic lateral sclerosis (ALS), epilepsy, and combination thereof. In some embodiments, the neurodegenerative disease is Alzheimer’s Disease.

Further features and advantages of the presently-disclosed subject matter will become evident to those of ordinary skill in the art after a study of the description, figures, and non-limiting examples in this document.

### BRIEF DESCRIPTION OF THE DRAWINGS

The presently-disclosed subject matter will be better understood, and features, aspects and advantages other than those set forth above will become apparent when consideration is given to the following detailed description thereof. Such detailed description makes reference to the following drawings, wherein:

FIG. 1 shows an image illustrating glycogen architecture: α-1,6-glycosidic bonds between glucose form the branch point for glycogen. Chain length is defined by α-1,4-glycosidic bonds glucose molecules. Phosphate can be added at C2-, C3- and C6-positions of glucose P.

FIG. 2 shows a schematic illustrating that glycogen is a critical component of cerebral metabolism. Glycogen metabolism is a direct contributor to the astrocyte-neuronal lactate shuttle that contributes to neuronal energy, while also providing a glucose energy reserve. Disruptions in glycogen metabolism alter synaptic activity, cognitive function and memory formation.

FIGS. 3A-D show images and graphs illustrating accumulation and properties of AD-PGBs. (A) Left, PAS staining of human AD brain slices showing accumulation of AD-PGBs in AD subjects. Arrows indicate AD-PGBs. Right, IHC showing Tau aggregation near the same region of AD brain. Scale bar = 5 µm. (B) Iodine staining showing increased chain length and branching in AD-PGBs. (C) Total phosphate content in purified PGBs, AD-PGBs, and normal glycogen. (D). Schematics of AD-PGBs vs. normal. Values shown are mean ± SEM of 30 patients. * p ≤ 0.05, ** p ≤ 0.01, *** p ≤ 0.001; two-tailed t-test.

FIG. 4 shows schematics of normal glycogen metabolism vs. step-wise formation of Alzheimer’s disease (AD)-polyglucosan bodies (PGBs). Early perturbation in glycogen synthesis or degradation leads to late stage abnormal branching and phosphate content that results in PGB/AD-PGBs formation. —> possible dysregulated pathways. GYS: glycogen synthase; BE: Branching enzyme; GDE: debranching enzyme; GP: glycogen phosphorylase; LA: laforin.

FIG. 5 shows schematics of muscle, liver, and AD-PGBs architecture, and their interactions with AMPK. Brain and muscle glycogen interact with AMPK via the β-subunit. Higher branched and phosphorylated muscle glycogen inhibits AMPK activity.

FIG. 6 shows a schematic illustrating a summary of the approach discussed in Example 1. A combination of in vitro and in vivo methods and models are used to investigate glycogen metabolism in AD.

FIGS. 7A-D show images and graphs illustrating methods of measuring GYS and GP activity. (A) Schematic of ¹³C-glycogen enrichment to measure GYS activity. (B) Schematic of “wash out” experiments to measure GP activity. (C) Representative image of isolated primary cortical astrocytes. (D) Rates of glycogen synthesis and degradation in primary cortical astrocytes.

FIGS. 8A-B show graphs illustrating correlation between glycogen content and Braak staging/Relative free glucose concentration. (A) Increased glycogen content from AD patient samples correlates with worse prognosis based on Braak staging. (B) Increase in total glycogen correlates with decreased free glucose in the brain from the same AD patient sampels as in A. R2 =0.36. 30 human autopsy samples from the UKY Brain Bank were used in both analyses. *p ≤ 0.05, **p ≤ 0.01, ***p ≤ 0.001; two-tailed t-test.

FIGS. 9A-B show graphs and images illustrating glycogen accumulation in multiple mouse models. (A) GCMS quantitation of glycogen content in all 3 AD mouse models vs. WT control confirming glycogen accumulation. 6-month-old mice were used for all groups. * p ≤ 0.05, ** p ≤ 0.01, *** p ≤ 0.001; two-tailed t-test. (B) 20x scan of IHC slides showing PGB accumulation in 5xFAD, rTg4510, and APOE4 mouse brains vs. WT. Dark brown stains are PGBs. 4x magnification for each brain are inserted in their respected images.

FIG. 10 shows a schematic illustrating a summary of the approach for Example 2: Age-related changes in glucose flux are studied in aging WT, 5xFAD, rTg4510 and APOE4 mice in vivo, as well as how these changes relate to AD pathology and progression. How AD-PGBs can modulate glucose metabolism through AMPK in vitro is also investigated.

FIGS. 11A-C show graphs and images illustrating glycogen content, glycogen phosphate content, and differences in glucose metabolic pathways. (A) GCMS quantitation of AD-PGBs purified from WT and 5XFAD mice at 8 and 16 weeks. (B) GCMS quantitation of phosphate content from (A). (C) Heatmap plot targeted metabolomics profiling showing differences early glucose metabolic pathways in WT and 5XFAD mice at 8 and 16 weeks (values are log transformed). * p < 0.05, ** p ≤ 0.01, *** p ≤ 0.001; two-tailed t-test.

FIGS. 12A-D show images illustrating cell-free glycogen binding assay. (A) Schematic of the cell-free glycogen AMPK binding assay - glycogen/AMPK complexes were captured by ConA conjugated to magnetic beads. (B) AMPK-WT has increased binding affinity to AD-PGBs. (C) AMPKβ_{T148D} mutant failed to bind to all glycogen types. (D) AMPKβ_{T148A} shows similar binding affinity to liver and AD-PGBs.

FIGS. 13A-D show images illustrating cell-free AMPK activity assay using ACC₁₋₁₅₀ : (A) Schematic of the cell-free AMPK activity assay. (B) Binding of AD-PGBs greatly reduces AMPK activity. (C) AMPKβ_{T148D} retains AMPK activity even after binding with AD-PGBs. (D) AMPKβ_{T148A} closely mimics WT-AMPK activity after binding to AD-PGBs.

FIGS. 14A-B show graphs illustrating solubility and chain length of glycogen. (A) Biochemical quantitation of soluble v. insoluble glycogen in control and branching enzyme (BE) knockdown in immortalized cortical astrocytes (ICE). (B) Iodine staining showing increased chain length and branching in ICA after BE knockdown. AD-G: AD-PGBs. * p ≤ 0.05, ** p ≤ 0.01, *** p ≤ 0.001; two-tailed t-test.

FIGS. 15A-G show a schematic and graphs illustrating a tracing of glucose carbon. (A) Tracing of glucose carbon through glycolysis and the Krebs cycle. Not all possible labeled metabolites are shown due to space limitations. : ¹²C; •,•: ¹³C from glycolysis, pyruvate dehydrogenase (PDH) and pyruvate carboxylase (PCB)-initiated reactions, respectively. (B) Decreased flux through glycolysis. (C-E) Decreased flux through the Krebs cycle were observed after shBE. The x-axis denotes the [number] of ¹³C atoms present in each compound. Values shown are mean ± SEM (average of 3 cell lines, n = 3, independent experiment). (G) Immunoblotting showing decreased AMPK activity after BE knockdown. * p ≤ 0.05, ** p ≤ 0.01, *** p ≤ 0.001; two-tailed t-test.

FIG. 16 shows a schematic illustrating a summary of the approach for Example 3: AD-PGBs is cleared in vivo using an antibody-enzyme fusion approach via ICV administration. Preclinical assessments to define the efficacy of treatment includes behavior, cerebral glucose metabolism, and neuropathology.

FIGS. 17A-C show graphs and images illustrating design of VAL-0417. (A) Pancreatic α-amylase has the highest activity under neutral pH. (B) Molecular design of VAL-0417, a fusion of the 3E10 Fab and α-amylase. (C) Schematic for the ELISA design to measure VAL-0417 limit of detection and standard curve to define linear range.

FIGS. 18A-D show graphs and images illustrating glycogen metabolism with VAL-0417 treatment in WT and Epm2a-/- (Lafora disease) mice. (A) Total brain glycogen (polysaccharide) normalized to tissue weight from untreated, PBS treated, and VAL-0417 treated. (B) VAL-0417 levels in PBS and VAL-0417 treated animals determined by ELISA. Data shown are the mean ± SE. The numbers of animals in each treatment group (n) are shown. * p ≤ 0.05, ** p ≤ 0.01, ***p ≤ 0.001. (C) PAS-stained brain slices of PBS and VAL-0417 treated Epm2a-/- mice after 12-day ICV infusion. White arrow indicates glycogen deposits that appear pinkish-purple; tissue was counterstained with hematoxylin (blue). (D) PCA plot of metabolite concentrations (>200) showing metabolism returning to normal after VAL-0417 treatment

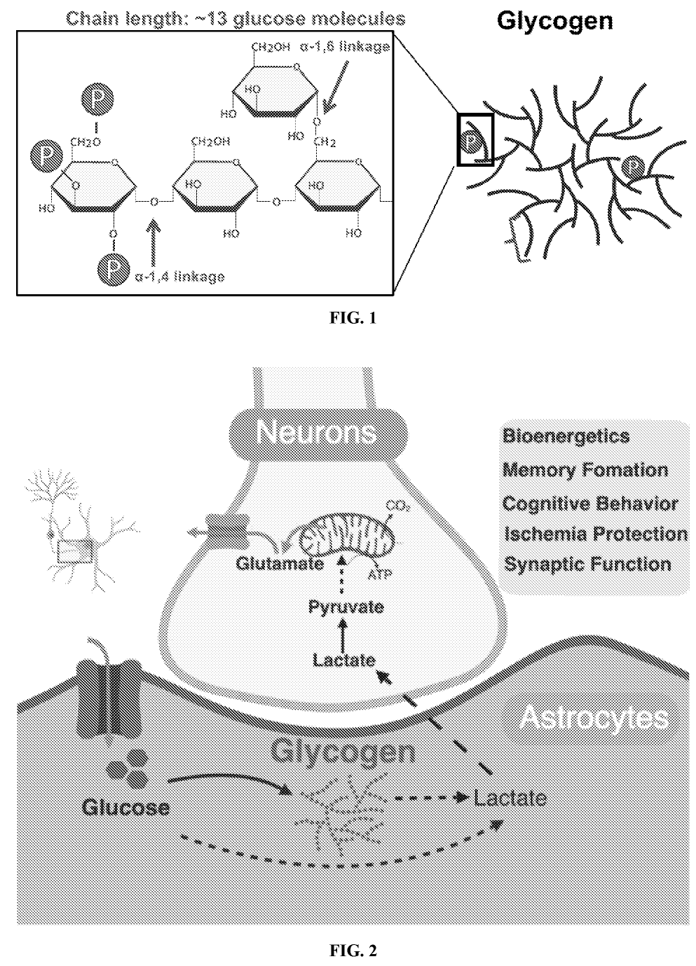

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DEFINITIONS

Unless defined otherwise, all technical and scientific terms used herein have the same meaning as commonly understood by one of ordinary skill in the art to which the disclosure belongs. Any methods and materials similar to or equivalent to those described herein can be used in the practice or testing of the present disclosure, including the methods and materials are described below.

Following long-standing patent law convention, the terms “a,” “an,” and “the” refer to “one or more” when used in this application, including the claims. Thus, for example, reference to “a cell” includes a plurality of cells, and so forth.

The terms “comprising,” “including,” and “having” are intended to be inclusive and mean that there may be additional elements other than the listed elements.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration, percentage, or the like is meant to encompass variations of in some embodiments ±50%, in some embodiments ±40%, in some embodiments ±30%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

All combinations of method or process steps as used herein can be performed in any order, unless otherwise specified or clearly implied to the contrary by the context in which the referenced combination is made.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E1Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

As used herein, the terms “optional” or “optionally” means that the subsequently described event or circumstance can or cannot occur, and that the description includes instances where said event or circumstance occurs and instances where it does not.

As used herein, the term “patient” refers to a subject afflicted with a disease or disorder. A patient includes human and veterinary subjects.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. It is also contemplated that, in certain aspects, unless expressly indicated to the contrary, individual substituents can be further optionally substituted (i.e., further substituted or unsubstituted).

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

### DETAILED DESCRIPTION

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

Provided herein are compounds for treating neurodegenerative diseases. In some embodiments, the compound includes one or more small molecule inhibitor and/or antisense oligonucleotide (ASO) that targets glycogen synthase (GYS) or other genes to down-regulate glycogen synthesis. Suitable small molecule GYS inhibitors include, but are not limited to:

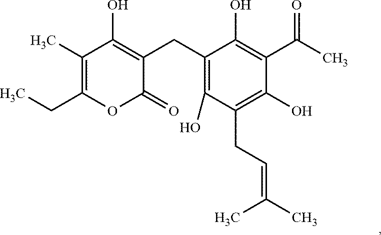

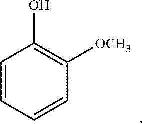

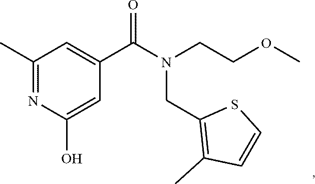

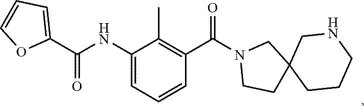

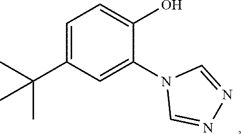

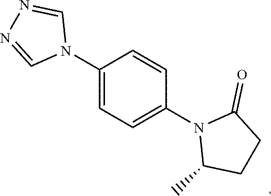

or a combination thereof. As will be understood by those skilled in the art, ASOs are single stranded nucleic acids that bind to complementary RNA sequences to down-regulate mRNA expression. Accordingly, suitable ASOs include any single stranded nucleic acid sequence that is complementary to a nucleic acid sequence encoding for GYS, such as mRNA coding for brain-expressed GYS. As will also be understood by those skilled in the art, ASO sequences targeting mRNA coding for GYS, such as Gys1-ASO, are readily designed and developed based upon the DNA sequence of GYS shown in SEQ ID NO:1. In some embodiments, one or more of the small molecule inhibitors and/or ASOs disclosed herein prevent polyglucosan body (PGB) formation in the brain. In some embodiments, one or more of the small molecule inhibitors and/or ASOs disclosed herein prevent PGB formation in the cytoplasm.

Additionally or alternatively, in some embodiments, the compound includes one or more antibody-enzyme fusion compounds. In one embodiment, the compound clears already formed polyglucosan-like granules in the brain. In some embodiments, the antibody-enzyme fusion includes the cell-penetrating antibody 3E10 linked to amylase or any suitable amylase variant. For examples, suitable antibody-enzyme fusion compounds include, but are not limited to, VAL1221, VAL0417, or a combination thereof. VAL1221 is a fusion protein comprising the Fab portion of the cell-penetrating antibody 3E10 linked to recombinant human acid alpha glucosidase (rhGAA). VAL0417 is a fusion protein comprising the cell-penetrating antibody 3E10 linked to amylase. In some embodiments, one or more of the antibody-enzyme fusion compounds disclosed herein clear and/or eliminate previously formed PGB in the brain. In some embodiments, one or more of the antibody-enzyme fusion compounds disclosed herein clear and/or eliminate previously formed PGB in the cytoplasm.

Also provided herein are methods for treating neurodegenerative diseases. In some embodiments, the method includes administering one or more of the compounds disclosed herein to a subject in need thereof. In some embodiments, the method includes administering one or more antibody-enzyme fusion compounds to clear PGBs already formed in the brain. For example, in one embodiment, the method includes administering VAL1221, VAL0417, or a combination thereof to clear PGBs already formed in the cytoplasm. Additionally or alternatively, in some embodiments, the method includes administering one or more small molecule inhibitors and/or ASOs to reduce or prevent PGB formation in the brain. For example, in one embodiment, the method includes administering one or more small molecule inhibitors and/or ASOs disclosed herein to reduce or prevent PGB formation in the cytoplasm. In another embodiment, the method includes first administering one or more antibody-enzyme fusion compounds to clear PGBs already formed in the brain, then administering one or more small molecule inhibitors and/or antisense oligonucleotides (ASO) to reduce or prevent glycogen/PGB formation in the brain, or vice versa. In some embodiments, in contrast to existing methods for treating glycogen storage disease (GSD), which include enzyme therapy targeting PGBs in the lysosomes, the methods disclosed herein target PGBs in the cytoplasm. Accordingly, without wishing to be bound by theory, it is believed that the methods disclosed herein represent treatment of neurodegenerative diseases through a novel, previously untested target.

The neurodegenerative disease according to the compounds and methods disclosed herein includes any suitable neurodegenerative disease involving abnormal accumulation of glycogen, such as accumulation of PGB in the brain. Suitable neurodegenerative diseases include, but are not limited to, traumatic brain injury (TBI), Alzheimer’s Disease (AD), amyotrophic lateral sclerosis (ALS), epilepsy, aging or a combination thereof. For example, in some embodiments, the methods disclosed herein include administering one or more of the compounds disclosed herein to treat early and/or late on-set AD. Additionally or alternatively, the compounds and methods disclosed herein may be anti-aging (i.e., the compounds and methods reduce, eliminate, or reverse the effects of aging, such as, but not limited to, the effects of aging on the brain).

Further provided herein, in some embodiments, is a method for stable isotope resolved metabolomics. In some embodiments, to assess glycogen synthesis, cells are isolated and matured, then ¹³C-glucose enriched culture media is added. The samples are then periodically collected to purify glycogen, and the fraction of labeled ¹³C-glycogen is measured over time. For example, to assess glycogen synthesis in cortical astrocytes, the cells are isolated and matured for 20 days. On the 21^{st} day, the ¹³C-glucose enriched culture media is added and the samples are collected every hour for eight hours to purify glycogen, and the fraction of labeled ¹³C-glycogen is measured over time. In some embodiments, to assess glycogen degradation, a ¹³C-glucose “wash out” experiment is performed. First, cells are cultured with ¹³C-glucose enriched media to achieve isotopic steady state of ¹³C-glycogen. Next, ¹³C-glucose media is replaced with regular ¹²C-glucose media and samples are periodically collected. Washout of ¹³C-glycogen is then measured over 8 hours to define the rate of glycogen degradation. For example, to assess glycogen degradation in primary astrocytes, the cells are cultured with ¹³C-glucose enriched media for 8 hours to achieve isotopic steady state of ¹³C-glycogen. ¹³C-glucose media is replaced with regular ¹²C-glucose media and samples are collected every hour for 8 hours. Washout of ¹³C-glycogen is measured over 8 hours to define the rate of glycogen degradation.

In some embodiments, the metabolomics method is used to establish age-related changes in cerebral glucose metabolic flux, aberrant glycogen formation, and AD related neuropathology in vivo across the lifespan of WT and multiple AD mouse models. In some embodiments, the metabolomics method is used to express mutant AMPK that cannot bind to AD-PGBs in both WT and AD-PGBs carrying immortalized astrocytes to define metabolic flux of glucose through central carbon metabolism and fatty acid biosynthesis controlled by AD-PGBs in vitro. Additionally or alternatively, in some embodiments, isotopologue distribution adds additional layers of information compared to traditional metabolomics approaches. For instance, by following isotope distribution of each metabolite, enzyme activity may be assessed and differential pathway contributions to the total metabolite pool may be calculated.

The presently-disclosed subject matter is further illustrated by the following specific but non-limiting examples. The following examples may include compilations of data that are representative of data gathered at various times during the course of development and experimentation related to the presently-disclosed subject matter. Those skilled in the art will recognize, or be able to ascertain, using no more than routine experimentation, numerous equivalents to the specific substances and procedures described herein.

### EXAMPLES

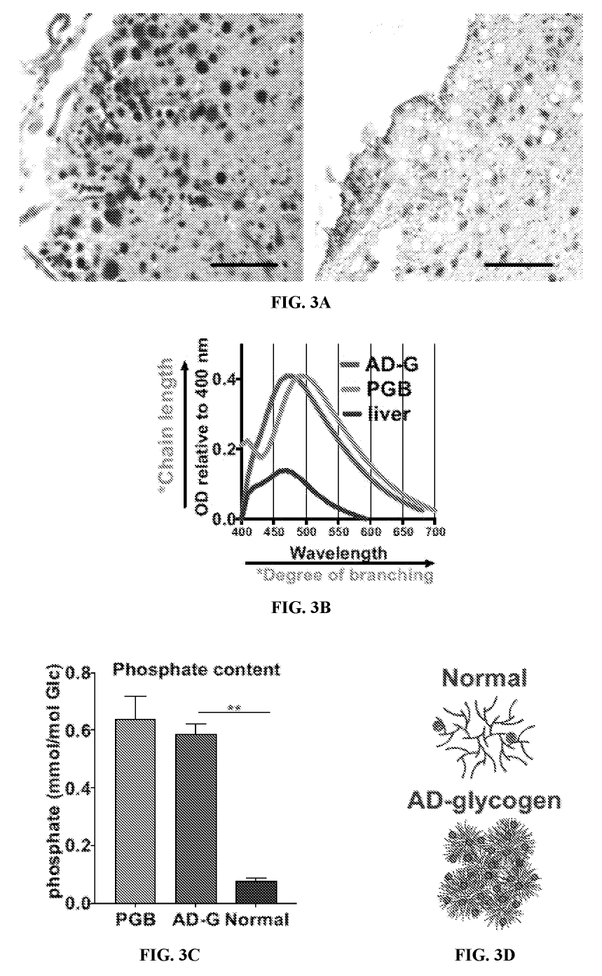

These Examples focus on the centralized theme that the accumulation of polyglucosan body (PGB)-like glycogen granules in Alzheimer’s disease (AD) brains, referred to herein as “AD-PGBs,” and their modulation of glucose metabolism are part of AD progression. AD-PGBs are up to 50 µm in diameter and are found in astrocyte cytoplasm, axons, and dendrites of the cerebral cortex in AD patients. AD-PGBs were found to be a universal feature in all 30 human brain specimens tested to date. Moreover, they are architecturally similar to GSD PGBs with respect to increased chain length and higher phosphate content (FIGS. 3A-D). Although previous reports have highlighted the pathogenesis of PGBs in cellular physiology, they have not established a connection between AD-PGBs with AD progression and physiopathology.

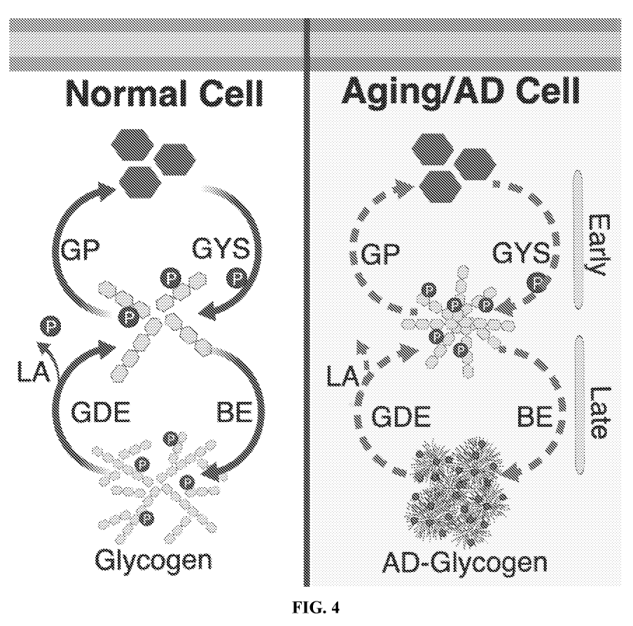

Based upon the results of the Examples presented herein, and without wishing to be bound by theory, it is believed that AD-PGBs formation follows a two-stage stepwise dysregulation in glycogen metabolism (FIG. 4). First, shifts in glycogen synthesis or degradation drive increases in glycogen size and phosphate content, which occurs early on during aggregate formation. Second, changes in glycogen branching enzyme, glycogen debranching enzyme and the glycogen phosphatase laforin results in the formation of AD-PGBs in neurological diseases. Following formation in the brain, AD-PGBs modulates cerebral glucose metabolism through direct binding and inhibition of AMP-activated protein kinase (AMPK).

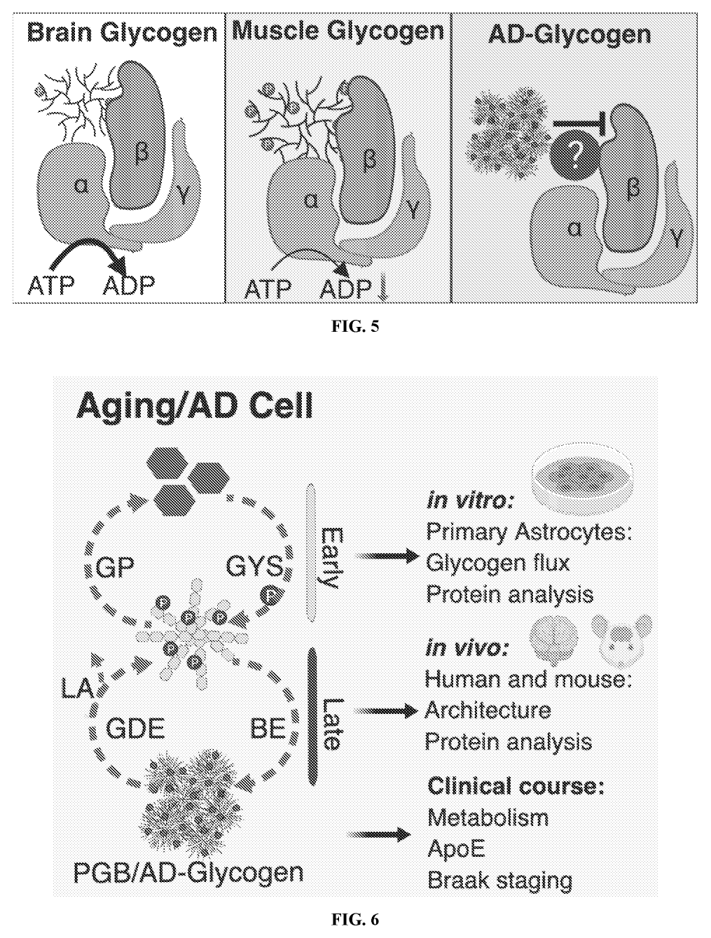

AMPK is a master regulator of glucose metabolism in multiple tissues including the brain. AMPK regulates glucose homeostasis by: 1) regulating glucose uptake, 2) maintaining the bioenergetic balance between glycolysis and mitochondrial metabolism, and 3) switching between fatty acid biosynthesis and oxidation. In mammals, AMPK exists as a heterotrimer (AMPKαβγ), containing a catalytic subunit (α) and regulatory subunits (β and γ) (FIG. 5). The β subunit of AMPK (AMPKβ) specifically binds to glycogen, forming a glycogen/AMPK complex (FIG. 5). Importantly, glycogen architecture modulates AMPK activity. For example, muscle glycogen has longer chain length, increased branching, and phosphate levels compared to brain glycogen. These characteristics result in greater binding affinity between muscle glycogen and AMPK, and this binding inhibits AMPK activity (FIG. 5). Again, without wishing to be bound by theory, it is believed that hyperbranched and hyperphosphorylated AD-PGBs has an enhanced inhibitory effect on AMPK, thereby acting as a driver of glucose hypometabolism during AD.

Since aberrant glycogen also accumulates in the aging brain, these findings have broader implications relating to the dynamics of cerebral metabolism and the aging process. In this regard, these Examples also define aging-associated metabolic changes in WT and AD mouse models using state-of-art approaches. More specifically, state-of-art techniques are applied to track cerebral glucose and glycogen metabolism perturbations in aging WT and multiple AD mouse models. Since there are definitive sex discrepancies regarding metabolism, cognition, and AD risk, power analysis and experimental procedures are designed to test sex as a biological variable.

Finally, these Examples explore whether clearance of AD-PGBs using the antibody-enzyme fusion VAL-0417 that ablates neuronal GSD PGBs is an effective therapeutic option to rescue glucose hypometabolism and cognitive function in aged WT and AD mouse models.

### Example 1 - Defining Glycogen Metabolism and AD-PGB Architecture in AD Models.

The accumulation of large insoluble glycogen-like polyglucosan bodies (PGBs) has been linked to increased ER stress, decreased proteasomal activity, increased misfolded proteins, and decreased autophagy in neurological GSDs. Strikingly, these are also all features of AD. While no direct link between glycogen and AD had previously been established, this Example shows that AD-PGBs are architecturally similar to PGBs and a universal feature of all AD autopsy specimens tested to date (n=30). The data provided in this Example demonstrates that AD-PGBs are both hyperbranched and hyperphosphorylated, similar to PGBs. Further, increased AD-PGBs content correlates with higher Braak scores and lower cerebral glucose. Finally, 5xFAD and rTg4510 AD mouse models, as well as human APOE4 “knock-in” mice all accumulate AD-PGBs. In view thereof, without wishing to be bound by theory, it is believed that AD-PGBs plays a pivotal role in AD disease progression. Therefore, understanding its biological origin is crucial for the future development of therapeutic interventions. To this end, innovative techniques are utilized to study glycogen metabolism, turnover, and architecture (FIG. 6).

### Methods

Flux analysis of glycogen metabolism in astrocytes. The biosynthesis and breakdown of glycogen involves glycogen synthase (GYS) and glycogen phosphorylase (GP), respectively. Early glycogen accumulation results in 1) an increase in synthesis and/or 2) a decline in degradation. Both enzymes are subject to post-translation modifications, and thus protein and mRNA levels do not always represent enzyme activity.

While traditional metabolomics are excellent at quantifying changes in metabolites after a specific perturbation, it is hard to interpret pathway activity or flux of a specific substrate such as glucose. For example, a decrease in a metabolite concentration can be indicative of increased consumption or decreased synthesis. Accordingly, stable isotope resolved metabolomics is more optimal over traditional metabolomics for these Examples. Only labeling pathways or metabolites directly downstream of glucose provides a highly-specific method to study glucose metabolism. Additionally, isotopologue patterns created from ¹³C labeled metabolites reveal information in enzyme activity and divergent pathways, providing an additional layer of information for functional interpretation. Furthermore, although the delivery of ¹³C-glucose to the brain has been a major barrier preventing in vivo stable isotope resolved metabolomics studies, these Examples employ a stress-free liquid diet delivery method specifically designed to study tissue glucose flux, including in the brain.

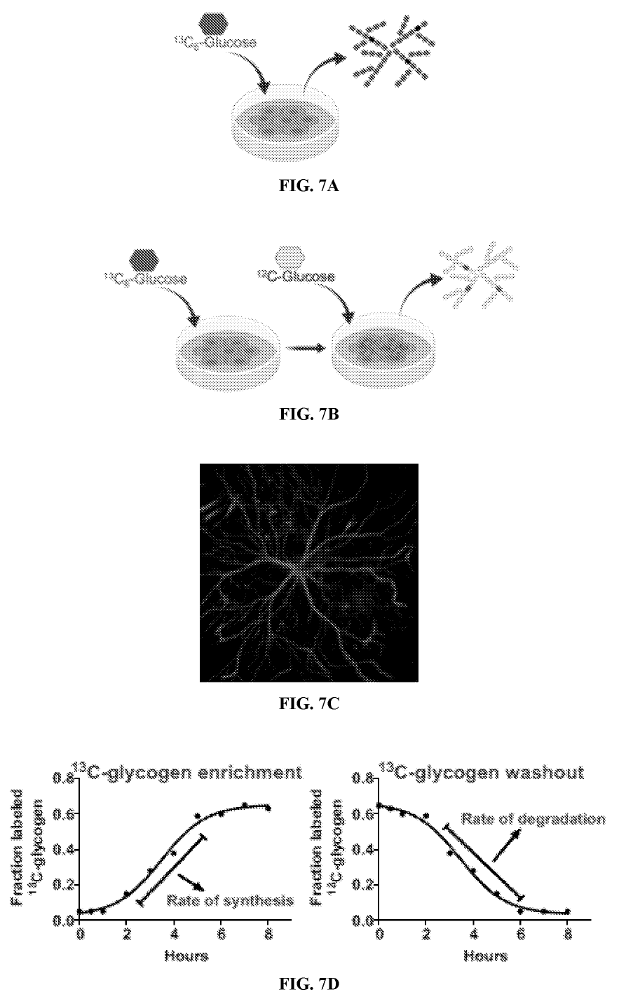

In view thereof, stable isotope resolved metabolomics that traces ¹³C-glucose metabolism through central carbon metabolism of the brain may be used to define cerebral glucose flux. For a true assessment of glycogen synthesis and degradation, flux analysis of heavy labeled glucose incorporation into glycogen over time is required to assess synthesis (FIG. 7A). Conversely, a ¹³C-glycogen “wash out” experiment can be used to assess the rate of degradation (FIG. 7B). For in vitro analysis, primary cortical astrocytes are isolated from WT, 5xFAD, rTg4510, and APOE4 mouse models of AD (FIG. 7C). To assess glycogen synthesis, cortical astrocytes are isolated and matured for 20 days. On the 21^{st} day ¹³C-glucose enriched culture media is added, samples are collected every hour for eight hours to purify glycogen, and the fraction of labeled ¹³C-glycogen is measured over time. For glycogen degradation, a ¹³C-glucose “wash out” experiment is performed. First, primary astrocytes are cultured with ¹³C-glucose enriched media for 8 hours to achieve isotopic steady state of ¹³C-glycogen. ¹³C-glucose media is replaced with regular ¹²C-glucose media and samples are collected every hour for 8 hours. Washout of ¹³C-glycogen is measured over 8 hours to define the rate of glycogen degradation. To demonstrate feasibility, this assay was performed on WT primary cortical astrocytes (FIG. 7D).

Additionally, GYS and GP protein levels may be assessed by western blotting to add additional layers of information to the flux analysis. Together, these experiments reveal whether early perturbations in glycogen synthesis or degradation is affected during progression of AD.

Biochemically define AD-PGBs architecture. Beyond glycogen synthesis and degradation, glycogen architecture is also an important aspect of glycogen physiology. Glycogen chain length, branching, and phosphate content are regulated by the coherent synergy between branching enzyme (BE), debranching enzyme (GDE), and laforin that define its granular size, solubility, accessibility to cells as a glucose storage cache, and interaction with AMPK. Dysregulation of any of these three enzymes could result in AD-PGBs formation. Glycogen architecture may be defined with high-resolution methods to identify metabolic defects that leads to AD-PGBs formation, pathogenesis of AD-PGBs, and its contribution to AD disease progression.

AD-PGBs, metabolism, clinical course, and disease progression in patient samples. An analysis of AD patients suggested an age-associated accumulation of AD-PGBs that correlates with Braak staging.

### Results

AD patient specimens have architecturally abnormal glycogen. Periodic acid-Schiff (PAS) staining was performed on AD specimens obtained at autopsy to assess glycogen content. Large granular AD-PGBs co-localize with tau aggregation in human AD patients (FIG. 3A). AD-PGBs was further purified and analyzed for relative architectural abnormalities using enzyme-based biochemical methods. AD-PGBs contains 4-fold higher phosphate than normal liver glycogen and has increased chain length and degree of branching by iodine spectra analysis (FIGS. 3B-C). AD-PGBs architectural properties closely resemble the malignant PGBs purified from a neuronal GSD mouse model (FIG. 3D). Together, this data suggests fundamental changes in glycogen metabolism and architecture may contribute to AD pathogenesis.

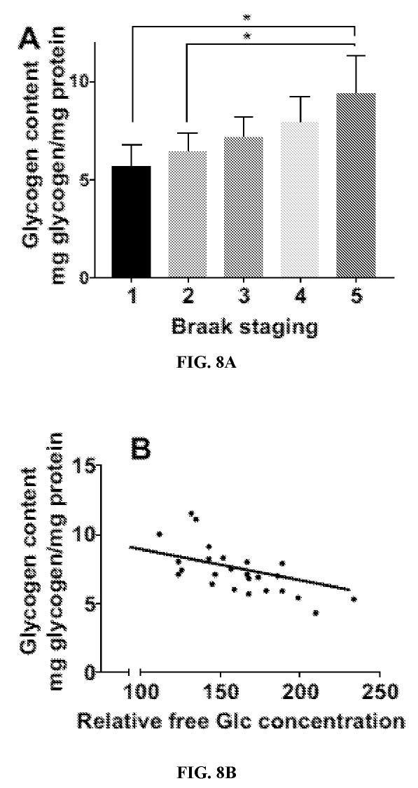

AD-PGBs correlate with Braak score and free cerebral glucose. To examine whether AD-PGBs correlate with AD progression, glycogen and free glucose were simultaneously purified from 30 patient samples (obtained from the UK Brain Bank with corresponding Braak staging) using a method that allows for sensitive quantification of both metabolites from as low as 10 mg tissue by gas chromatography-coupled mass spectrometry (GCMS). A strong positive correlation was found between AD-PGBs levels and Braak staging (FIG. 8A). Additionally, increases in AD-PGBs were found to correlate with decreased total pools of free glucose in the brain (FIG. 8B). These data suggest AD-PGBs modulates free glucose levels in the brain and represents a key aspect of AD disease progression.

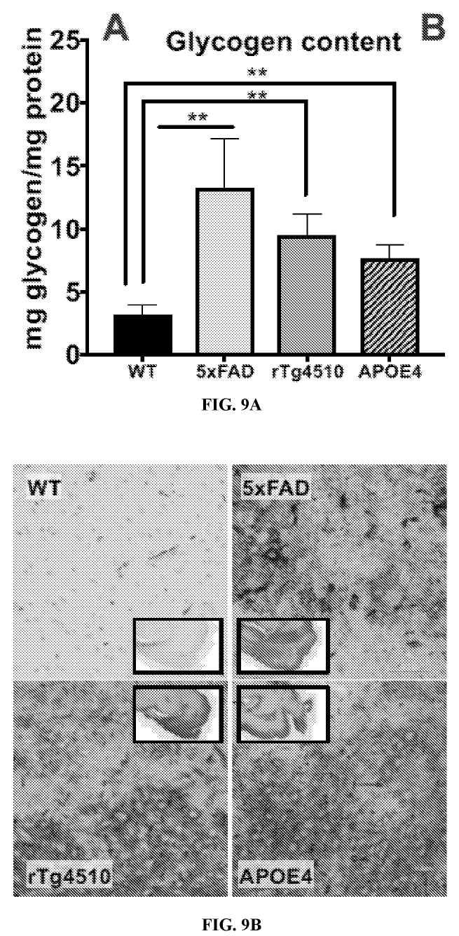

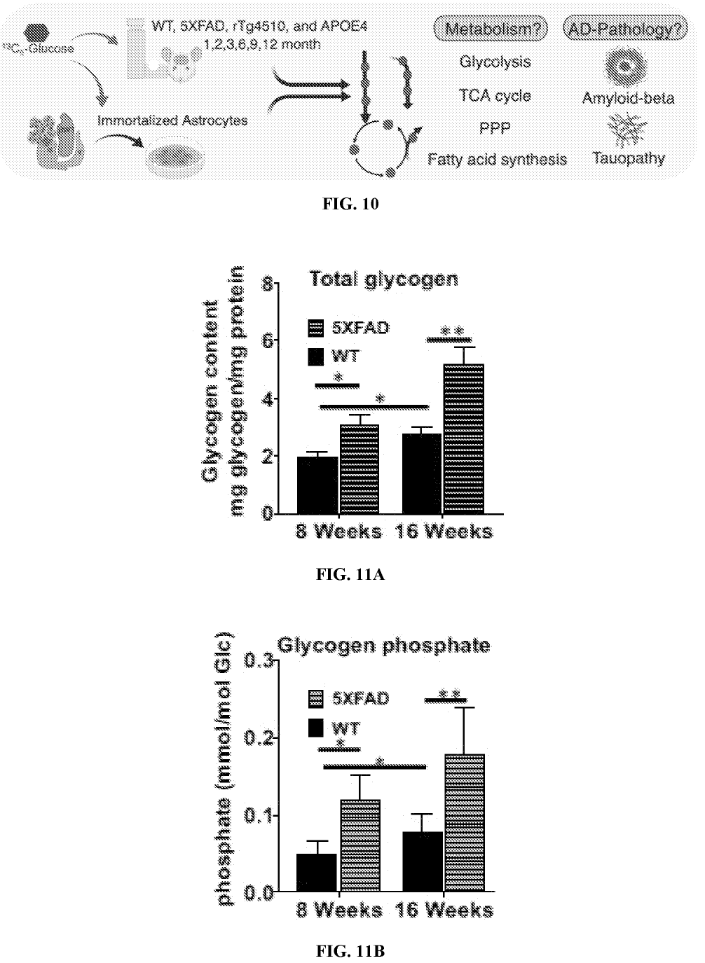

5xFAD, rTg4510, and human APOE4 transgenic mice all show accumulation of AD-PGBs. To assess if AD-PGBs exists in preclinical models, two different AD mouse models were utilized: the 5xFAD amyloid pathology model and the rTg4510 tauopathy model. Human APOE4 targeted replacement transgene mice (Apoe^{tm3}(^{APOE*4})^{Mae}) were also utilized, a model that shares metabolic characteristics with humans, but does not form amyloid-beta and tauopathy. Glycogen was purified from whole brain and quantified glycogen loads using GCMS and immunohistochemical staining (IHC). A more than 2-fold increase in glycogen accumulation was found in all three models compared to their age-matched WT controls (FIG. 9A). Moreover, AD-PGBs was clearly visible with IHC in sagittal brain sections using a highly specific PGB antibody (FIG. 9B). Importantly, the presence of AD-PGBs in the APOE4 mouse brain suggests that AD-PGBs is not simply a byproduct of amyloid or tau pathology, but instead may be a potential driver of the disease.

### Conclusion

This Example resolves the metabolic perturbations that result in AD-PGBs formation during disease progression. Defining AD-PGBs metabolism using flux analysis in three different AD mouse models permits a determination as to whether AD-PGBs arise from early increases in synthesis or reduction in degradation. AD-PGB architecture identifies unique properties of other glycogen modeling enzymes that drive disease progression.

The astrocyte analyses suggest ¹³C-glucose enrichment of glycogen reaches isotopic steady state at 8 hours. However, if glycogen metabolism is compromised in primary astrocytes isolated from AD mouse models, this time frame could be altered. If this is the case, enrichment and wash out experimental time points may be increased/decreased to carefully evaluate glycogen synthesis and degradation. Although astrocytes have been identified as the principal site of glycogen metabolism in the brain, possible glycogen accumulation has been observed in neurons as well.

### Example 2 - Determining Glycogen Modulation of Cerebral Metabolism in AD and Aging.

Age-related changes in metabolism with the onset and progression of AD have not been well established, and thus remain a knowledge gap in the field. This Example shows age-dependent changes in glycogen accumulation and glucose metabolism in both WT and 5xFAD mice. Moreover, this Example shows that AD-PGBs can directly bind and modulate AMPK activity. AMPK is a master regulator of metabolism - controlling glucose flux, glycolysis, mitochondrial respiration, and fatty acid biosynthesis in the brain. Therefore, without wishing to be bound by theory, it is believed that AD-PGBs modulates cerebral glucose metabolism through AMPK during aging and the progression of AD.

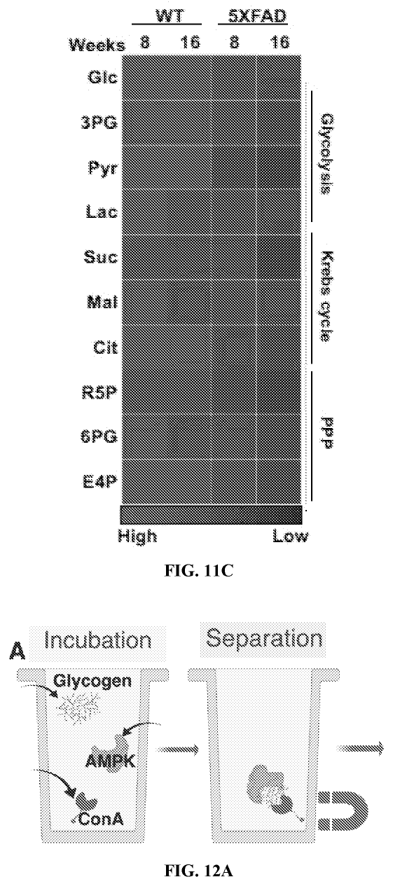

A novel stable isotope resolved metabolomics approach may be used to: 1) establish age-related changes in cerebral glucose metabolic flux, aberrant glycogen formation, and AD related neuropathology in vivo across the lifespan of WT and multiple AD mouse models; and 2) express mutant AMPK that cannot bind to AD-PGBs in both WT and AD-PGBs carrying immortalized astrocytes to define metabolic flux of glucose through central carbon metabolism and fatty acid biosynthesis controlled by AD-PGBs in vitro (FIG. 10).

### Methods

Define age-related changes in glycogen, glucose hypometabolism, and AD progression.

Clearly delineating age-related changes in AD-PGBs, glucose hypometabolism, amyloid pathology, and tauopathy would establish the relationship between them, as well as their individual roles in driving the onset and progression of AD. In this regard, the present inventors have developed an innovative non-invasive liquid diet that may be used to study in vivo glycogen and glucose flux. This method allows for stress-free stable isotope delivery and enrichment of all major metabolites — from nucleotides to fatty acids — in vivo. Two-week, one-, two-, four-, six-, nine-, and twelve-month-old age- and sex-matched WT, 5xFAD, rTg4510, and APOE4 mice are fed 680 g diet/kg mouse supplemented with 0.167 g ¹³C-glucose/g diet to allow ad libitum consumption for 18 hours (n=14 provides 85% power to detect 20% change in metabolites at 5% false discovery rate). While this liquid diet allows stress-free ad libitum feeding, if eating patterns are disrupted in AD mice it may not be applicable. As such, the diet is monitored carefully by weighing liquid diet daily. If the eating pattern is altered a newly developed gavage method may be used for consistent delivery of ¹³C-glucose across different age in WT and AD mice.

At the end of the feeding period, mice are sacrificed by spinal dislocation followed by immediate brain extraction. Brains are dissected to cortex, hippocampus, and cerebellum and snap frozen in liquid N₂. The frozen tissues from in vivo studies is pulverized to 10 µm particles in liquid N₂ using a magnetic assistant tissue milling and 20 mg of each milled tissue is extracted in a solvent mixture of H₂O:methanol:chloroform (1:1:2) for recovering polar, lipid, and glycogen. The polar extracts are then analyzed for a wide range of metabolites and their labeling patterns using GCMS. Measurements include downstream metabolites from central carbon metabolism including, glycolysis, PPP, Krebs cycle, amino acid metabolism, and metabolism of nucleotides and their derivatives. The organic layer is analyzed for ¹³C-enrichment of free fatty acid biosynthesis with variable chain lengths (C:8-C:40). The insoluble layer is further extracted using 10% TCA to isolate glycogen, and glycogen and phosphate content are assessed using methods in Example 1. ¹³C incorporation into metabolites downstream of ¹³C-glucose is followed in order to reconstruct metabolic networks specifically altered during normal aging and during the onset and progression of AD in mouse models. The remaining hemisphere of the brain is stained for amyloid-beta and tauopathy to better understand the impact of altered metabolism on the biology of aging and AD progression.

Together, these data establish the sequence of events connecting AD-PGB formation, AD-related neuropathology, and glucose metabolism. Further, by including a WT aging group, the experiments provide insight into natural aging events, and thus define the point of divergence between normal aging and AD-specific pathophysiology.

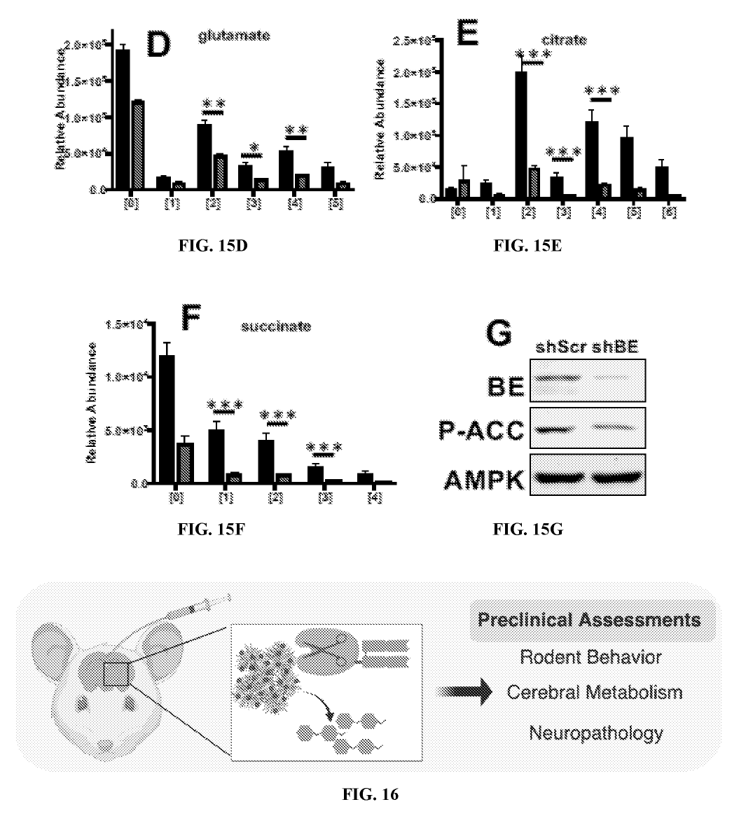

Identify metabolic perturbations driven by AD-PGBs inhibition of AMPK. Cells harboring shRNA targeting AMPKct have aberrant central carbon metabolism including glycolysis, Krebs cycle, and fatty acid synthesis. Since AD-PGBs completely inactivate AMPK in cell-free assays, it is believed that this reprograms cerebral glucose metabolism in a pathogenic manner. To interrogate detailed metabolic flux through different glucose metabolic pathways after AD-PGBs/AMPK interaction, a stable isotope resolved metabolomics approach is utilized in vitro. WT and mutant AMPKβ cell lines generated as discussed above are used to study glucose flux through central carbon metabolism. Isotopologue distribution adds additional layers of information compared to traditional metabolomics approaches. For instance, by following isotope distribution of each metabolite, enzyme activity may be assessed and differential pathway contributions to the total metabolite pool may be calculated (FIG. 15A).

### Results

Age-dependent accumulation of AD-PGBs and impairments in glucose hypometabolism. The 5xFAD mouse strain is a model of rapid amyloid pathology with cognitive deficits. To demonstrate age-related AD-PGBs formation and glucose hypometabolism, a glycogen analysis and targeted metabolomics were performed on WT and 5xFAD mouse brains at 8 and 16 weeks. Age associated increases in glycogen accumulation (FIGS. 11A-B), and decreases in glucose metabolism were found in 16-week old WT mice when compared to 8-week controls. However, the differences are exacerbated in the 5xFAD model (FIG. 11C), suggesting a more significant shift in metabolism is required for the progression of AD.

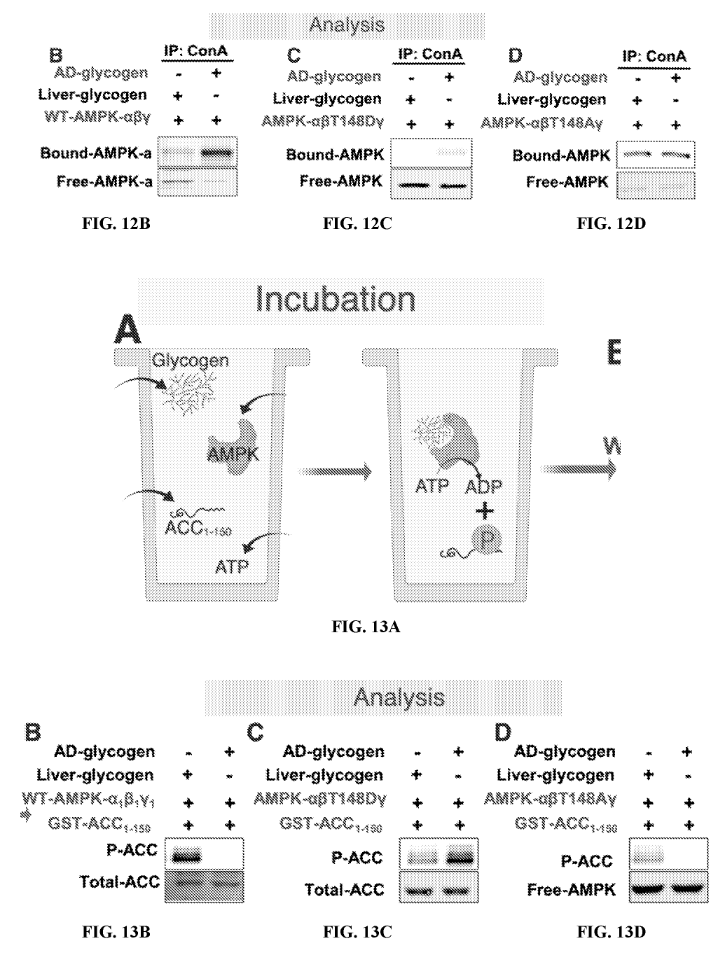

AMPK has increased binding affinity to AD-PGBs. AMPK modulates the rate of glucose uptake, glycolysis and fatty acid biosynthesis. AMPK activation in the brain is protective against focal cerebral ischemia and glucose deprivation, and loss of AMPK activity results in aberrant metabolism in experimental models. The β subunit of AMPK (AMPKβ) contains a conserved region called the carbohydrate binding module (CBM), and threonine 148 (AMPKβ_{T148}) of the CBM is a key amino acid for the glycogen/AMPK interaction. Substitution with the phosphomimic aspartic acid (AMPKβ_{T148D}) prevents AMPK binding to glycogen. Conversely, the AMPKβ_{T148A} mutant binds to glycogen with increased affinity. To study AMPK interaction with AD-PGBs, a cell-free assay was established using bacterially expressed recombinant wild type and mutant AMPK trimeric complexes, purified AD-PGBs from the brain of 5xFAD mice, and the glycogen-binding ConA conjugated to magnetic beads to capture the complex (FIG. 12A).

WT-AMPK binds to AD-PGBs with 7-fold higher affinity than liver glycogen isolated from the same mouse. As expected, the T148D mutant has limited binding affinity to AD-PGBs while the T148A control has the opposite effect showing comparable binding to both glycogen subtypes (FIGS. 12B-D). These data demonstrate that AD-PGBs interacts with AMPK with high affinity and the interaction is dependent on AMPKβ_{T148}.

Binding of AD-PGBs to AMPK abolishes its activity. To test whether binding to AD-PGBs impacts AMPK activity, recombinant AMPK complexes were co-expressed with AMPK-upstream activator CAMKKβ in bacteria to produce activated AMPK. The ability of AD-PGBs/AMPK complexes to phosphorylate a recombinant peptide corresponding to the 1-150 amino acid sequence of acetyl-CoA carboxylase (ACC₁₋₁₅₀), a known AMPK substrate (FIG. 13A), was tested. The binding to AD-PGBs resulted in the inability of AMPK to phosphorylate ACC₁₋₁₅₀ in the cell-free assay. This was rescued by the low affinity β_{T148D} mutant, but not the high affinity β_{T148A} mutant (FIGS. 13B-D). These data confirm that AMPK binds to AD-PGBs with high affinity and overrides AMPK activity. Together, these cell-free analyses confirm that the AMPK genetic mutants are powerful tools to study AD-PGBs biology in vitro.

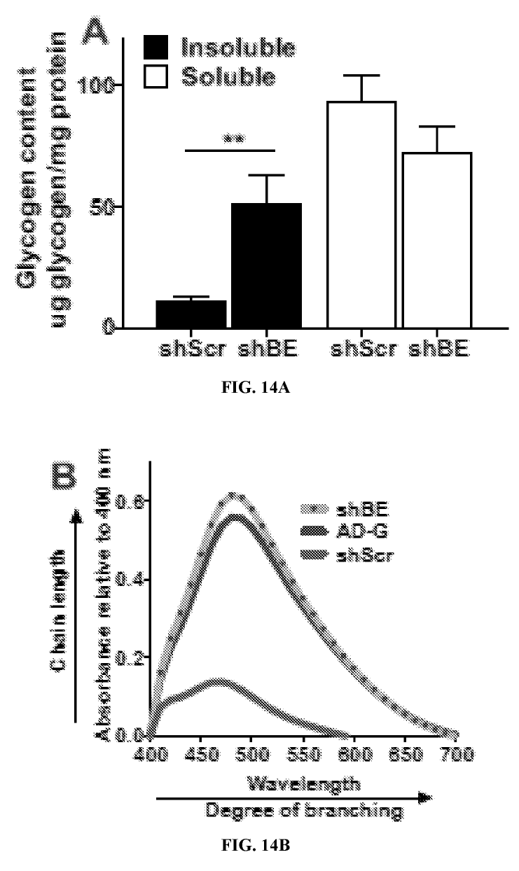

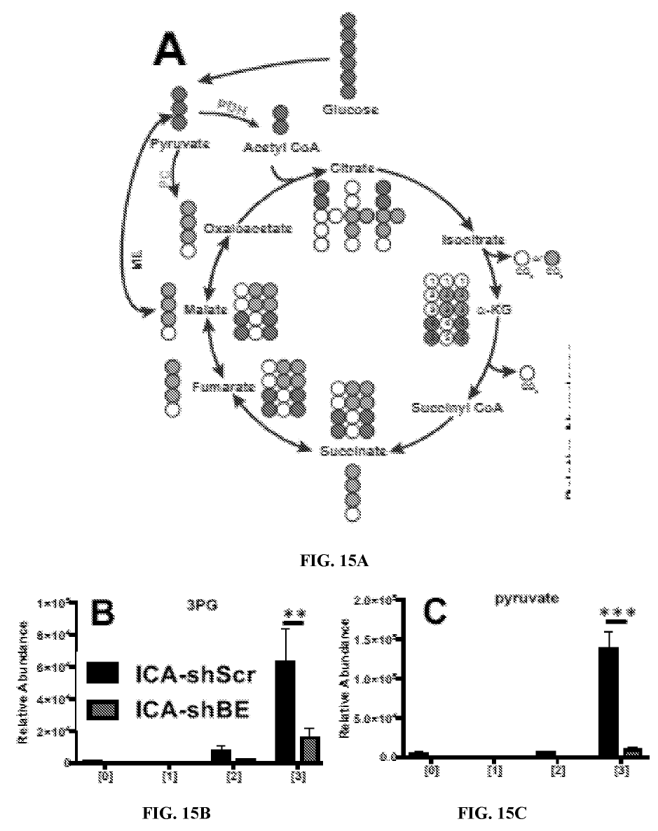

Generation of cell line with AD-PGBs. Astrocytes play critical roles in maintaining cerebral metabolism, and their dysregulation is thought to contribute to the pathogenesis of AD. Much of what is known about astrocyte biology is derived from in vitro studies of isolated human or rodent astrocytes. Currently, an astrocyte model that contain AD-PGBs does not exist. In order to study the impact of AD-PGBs on glucose metabolism in vitro, immortalized astrocytes were genetically engineered to accumulate AD-PGBs based on glycogen storage disease type IV (GSD-IV). GSD-IV results from glycogen branching enzyme (BE) deficiency. BE is required for the assembly of a branched glycogen molecule, which enables a high packing density of glucose for maximum storage. GSD-IV results in systemic aberrant insoluble glycogen accumulation (similar in structure to AD-PGBs) that drives neurodegeneration in cats and horses. A stable knockdown of BE using small hairpin RNA was performed in immortalized cortical astrocytes (ICA), using scrambled shRNA(shScr) as control. ICA-shBE contain 5-fold higher insoluble glycogen than controls (FIG. 14A), and the glycogen is hyperbranched and hyperphosphorylated similar to AD-PGBs (FIGS. 14A-B). ICA-shBE is used as an in vitro model of AD-PGBs accumulation to interrogate its effects on AMPK and glucose metabolism.

In vitro AD-PGBs model has altered AMPK activity and glucose metabolism. To confirm that in vitro models of AD-PGBs in ICA modulate AMPK activity and glucose metabolism, immunoblotting and stable isotope resolved metabolomics analysis were performed on ICA-shBE and control (ICA-ShScr) cell lines. To investigate whether AD-glucose alters glucose metabolism, ¹³C₆-glucose was employed as a tracer to study carbon flux through glycolysis and the Krebs cycle in ICA-shBE and control cell lines (FIG. 15A). A decrease in glucose uptake and decreases in the relative amounts of metabolites and their isotopologues in glycolysis and the Krebs cycle were observed (FIGS. 15B-F). Additionally, ICM-shBE had reduced p-ACC compared to the control, suggesting inhibition of AMPK activity (FIG. 15G). These data confirm ICA-shBE have slower glucose metabolism, and AD-PGBs is a direct modulator of glucose metabolism in astrocytes.

### Conclusion

The data obtained through the Examples discussed herein show that AD-PGBs have a dominant inhibitory effect over AMPK in a cell-free assay. This Example confirms that the AD brain has altered metabolism and that AD-PGBs could drive this phenotype through inactivation of AMPK. Additionally, the results of the experiments discussed herein improve the understanding of age-associated metabolic changes in AD, thereby providing scientific rationale for the development of the next generation of anti-AD therapeutics.

### Example 3 - Targeting of AD-PGBs With an Antibody-Enzyme Fusion to Treat AD.

Abnormal glycogen accumulation is the defining etiology of multiple glycogen storage diseases (GSDs). Systemic enzyme replacement therapy ― which degrades glycogen ― relieves the clinical burdens of GSD type II. GSDs of the brain lead to rapid cognitive decline and accelerated neurodegeneration. No therapy exists to target abnormal glycogen in the brain. However, a conditional knock out of glycogen synthase in a brain GSD mouse model prevents glycogen formation, reverses many side-effects of aging, and reduces neuroinflammation and gliosis. In view thereof, it is believed that clearance of AD-PGBs in AD models would restore metabolic profiles, improve cognitive function, and delay neurodegeneration.

The present inventors have demonstrated the feasibility of intracerebroventricular (ICV) delivery of a novel antibody-enzyme fusion therapeutic, VAL-0417, to degrade abnormal glycogen of the brain in vivo. This delivery is used to assess whether VAL-0417 can improve AD behavioral/cognitive functions, neuropathology, and metabolism in 5xFAD, rTg4510, and APOE4 mouse models (FIG. 16).

### Methods

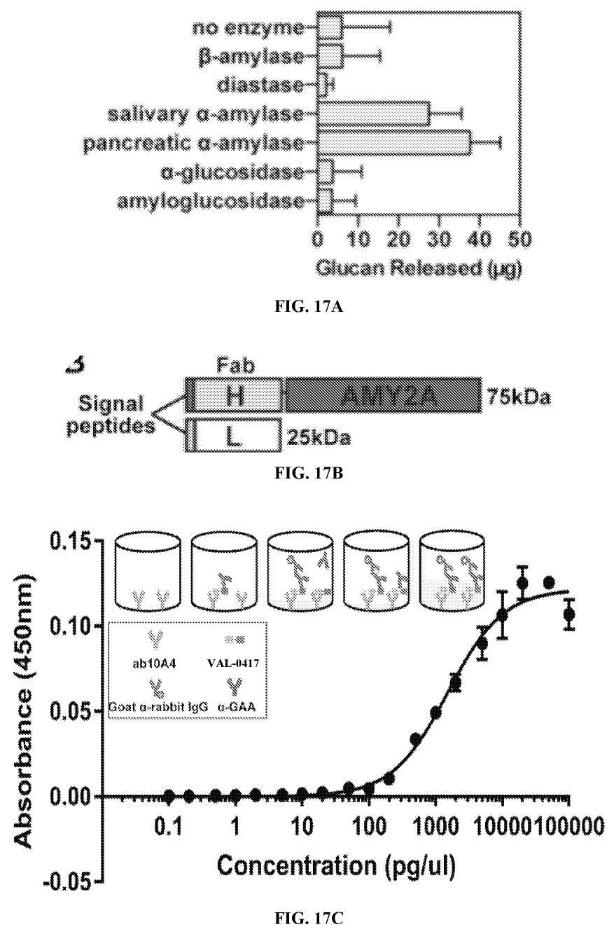

Antibody enzyme fusion targeting cerebral glycogen. Enzyme therapeutics hold a few advantages over small molecule inhibitors for the treatment of intracellular aggregates due to i) direct targeting, ii) substrate specificity, and iii) robust half-life in vivo. Genetically engineered antibody enzyme fusions (AEFs) improve on traditional enzyme therapeutics to allow targeted penetration of diseased tissues and cell types. The AEF designated VAL-0417 was generated by fusing the 3E10 antibody heavy chain fragment with human pancreatic α-amylase (FIGS. 17A-B). 3E10 can penetrate brain cells using a nearly ubiquitously expressed nucleotide salvage pathway mediated by the equilibrative nucleoside transporter 2 (ENT2). The human pancreatic α-amylase displayed the best activity to degrade glycogen under neutral pH of the cellular environment (FIG. 17A). VAL-0417 is designed as a treatment option delivered through intracerebroventricular injection (ICV). A highly sensitive enzyme-linked immunosorbent assay (ELISA) was developed to detect VAL-0417 as low as 50 pg VAL-0417/µg of protein (FIG. 17C). The ELISA provides a rapid method of detecting VAL-0417, it differentiates VAL-0417 from endogenous amylase proteins, and it provides a robust signal from multiple tissue.

Examine glucose flux, AMPK activity, bioavailability of VAL-4017 in vivo after treatment. Biodistribution of VAL-0417 is measured from samples using an in-house ELISA that has a sensitive of detection of 50 pg/ug protein (FIG. 17C).

### Results

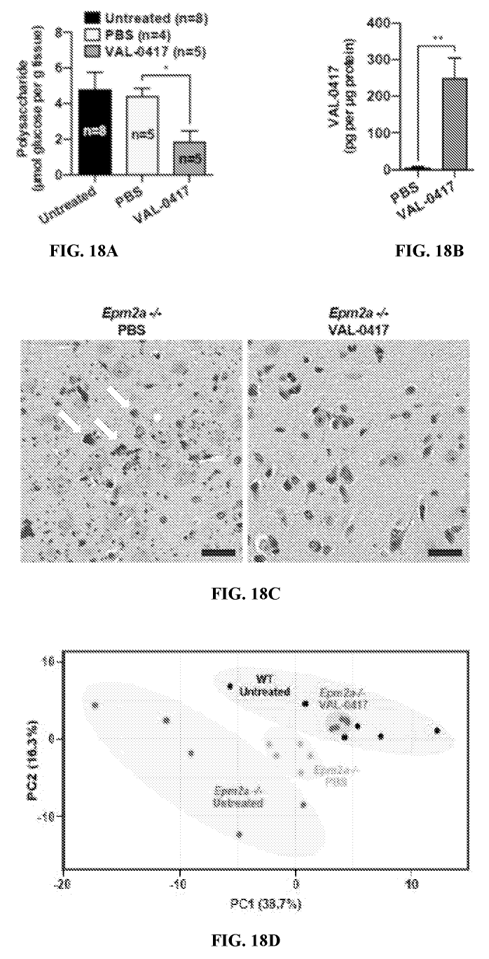

The AEF VAL-0417 degrades PGBs in the brain. The ability of VAL-0417 to degrade abnormal glycogen was first tested in a murine GSD model of neurodegeneration (Epm2a^{-/-}) in vivo. VAL-0417 was administered directly into the right lateral ventricle using an ALZET osmotic pump attached to a catheter that was surgically implanted for intracerebroventricular (ICV) delivery. 6-month-old mice received a continuous infusion of VAL-0417 (0.026 mg/day) or PBS for 12 days via the ICV pump. Mice were then euthanized, followed by assessment of total glycogen levels and the cerebral metabolome. Strikingly, VAL-0417 dramatically reduced total glycogen levels by greater than 70% (FIGS. 18A-C). The results demonstrated that VAL-0417 degrades brain PGBs after ICV administration.

While these results are extremely exciting, they do not demonstrate that eliminating PGBs impacts brain physiology. Therefore, GCMS was utilized to assess >150 brain central carbon metabolites of glycolysis and the TCA cycle; amino acids; nucleotides. The metabolic profile of untreated WT and Epm2a^{-/-} mice segregated into two very distinct groups (FIG. 18D). The metabolic profile from the PBS-treated laforin KO mice clustered into a third group, likely due to the physiological effects of surgery and PBS infusion (FIG. 18D). Strikingly, metabolites of the VAL-0417 treated laforin KO mice clustered tightly within the WT group, indicating that the cerebral metabolism returned to normal.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Narayan, K.M., et al., Impact of recent increase in incidence on future diabetes burden: U.S., 2005-2050. Diabetes Care, 2006. 29(9): p. 2114-6.

2. Gallagher, C.H., J.D. Judah, and K.R. Rees, Glucose oxidation by brain mitochondria. The Biochemical journal, 1956. 62(3): p. 436-440.

3. Hertz, L. and G.A. Dienel, Energy metabolism in the brain, in International Review of Neurobiology, D.S. Dwyer, Editor. 2002, Academic Press. p. 1-IN4.

4. Vannucci, R.C. and S.J. Vannucci, Glucose metabolism in the developing brain. Seminars in Perinatology, 2000. 24(2): p. 107-115.

5. Cunnane, S., et al., Brain fuel metabolism, aging, and Alzheimer’s disease. Nutrition, 2011. 27(1): p. 3-20.

6. Schurr, A., C.A. West, and B.M. Rigor, Lactate-supported synaptic function in the rat hippocampal slice preparation. Science, 1988. 240(4857): p. 1326-1328.

7. Magistretti, P.J. and L. Pellerin, Astrocytes couple synaptic activity to glucose utilization in the brain. Physiology, 1999. 14(5): p. 177-182.

8. Rangaraju, V., N. Calloway, and T.A. Ryan, Activity-driven local ATP synthesis is required for synaptic function. Cell, 2014. 156(4): p. 825-835.

9. Small, G.W., et al., Cerebral metabolic and cognitive decline in persons at genetic risk for Alzheimer’s disease. Proc Natl Acad Sci USA, 2000. 97(11): p. 6037-42.

10. Blass, J.P., Alzheimer’s disease and Alzheimer’s dementia: distinct but overlapping entities. Neurobiol Aging, 2002. 23(6): p. 1077-84.

11. Grady, C.L., et al., Stability of metabolic and neuropsychological asymmetries in dementia of the Alzheimer type. Neurology, 1986. 36(10): p. 1390-2.

12. Haxby, J.V., et al., Longitudinal study of cerebral metabolic asymmetries and associated neuropsychological patterns in early dementia of the Alzheimer type. Arch Neurol, 1990. 47(7): p. 753-60.

13. Altmann, A., et al., Regional brain hypometabolism is unrelated to regional amyloid plaque burden. Brain, 2015. 138(Pt 12): p. 3734-46.

14. Reiman, E.M., et al., Preclinical evidence of Alzheimer’s disease in persons homozygous for the epsilon 4 allele for apolipoprotein E. N Engl J Med, 1996. 334(12): p. 752-8.

15. Brown, A.M. and B.R. Ransom, Astrocyte glycogen and brain energy metabolism. Glia, 2007. 55(12): p. 1263-1271.

16. Hertz, L., L. Peng, and G.A. Dienel, Energy metabolism in astrocytes: high rate of oxidative metabolism and spatiotemporal dependence on glycolysis/glycogenolysis. J Cereb Blood Flow Metab, 2007. 27.

17. Walls, A.B., et al., Robust glycogen shunt activity in astrocytes: Effects of glutamatergic and adrenergic agents. Neuroscience, 2009. 158(1): p. 284-292.

18. Suzuki, A., et al., Astrocyte-Neuron Lactate Transport Is Required for Long-Term Memory Formation. Cell, 2011. 144(5): p. 810-823.

19. Attwell, D. and S.B. Laughlin, An energy budget for signaling in the grey matter of the brain. J Cereb Blood Flow Metab, 2001. 21(10): p. 1133-45.

20. Hertz, L., et al., Reciprocal changes in forebrain contents of glycogen and of glutamate/glutamine during early memory consolidation in the day-old chick. Brain Res, 2003. 994(2): p. 226-33.

21. Gibbs Marie, E., G. Anderson Damian, and L. Hertz, Inhibition of glycogenolysis in astrocytes interrupts memory consolidation in young chickens. Glia, 2006. 54(3): p. 214-222.

22. Tagliabracci, V.S., et al., Phosphate incorporation during glycogen synthesis and Lafora disease. Cell metabolism, 2011. 13(3): p. 274-282.

23. Contreras, C.J., et al., Incorporation of phosphate into glycogen by glycogen synthase. Archives of Biochemistry and Biophysics, 2016. 597: p. 21-29.

24. Worby, C.A., M.S. Gentry, and J.E. Dixon, Laforin, a dual specificity phosphatase that dephosphorylates complex carbohydrates. Journal of Biological Chemistry, 2006. 281(41): p. 30412-30418.

25. Gentry, M.S., C. Romá-Mateo, and P. Sanz, Laforin, a protein with many faces: glucan phosphatase, adapter protein, et alii. The FEBS journal, 2013. 280(2): p. 525-537.

26. Li, C., O. Powell Prudence, and G. Gilbert Robert, Recent progress toward understanding the role of starch biosynthetic enzymes in the cereal endosperm. Amylase, 2017. p. 59.

27. Powell, P.O., et al., Acid hydrolysis and molecular density of phytoglycogen and liver glycogen helps understand the bonding in glycogen alpha (composite) particles. PLoS One, 2015. 10(3): p. e0121337.

28. Deng, B., et al., The Mechanism for Stopping Chain and Total-Molecule Growth in Complex Branched Polymers, Exemplified by Glycogen. Biomacromolecules, 2015. 16(6): p. 1870-2.

29. Sharma, J., et al., Neuronatin-mediated aberrant calcium signaling and endoplasmic reticulum stress underlie neuropathology in Lafora disease. Journal of Biological Chemistry, 2013. 288(13): p. 9482-9490.

30. Hetz, C. and B. Mollereau, Disturbance of endoplasmic reticulum proteostasis in neurodegenerative diseases. Nature Reviews Neuroscience, 2014. 15(4): p. 233.

31. Sampaolo, S., et al., A novel GBE1 mutation and features of polyglucosan bodies autophagy in adult polyglucosan body disease. Neuromuscular Disorders, 2015. 25(3): p. 247-252.

32. Kakhlon, O., et al., Polyglucosan neurotoxicity caused by glycogen branching enzyme deficiency can be reversed by inhibition of glycogen synthase. Journal of neurochemistry, 2013. 127(1): p. 101-113.

33. Ganesh, S., et al., Transcriptional profiling of a mouse model for Lafora disease reveals dysregulation of genes involved in the expression and modification of proteins. Neuroscience letters, 2005. 387(2): p. 62-67.

34. Bernier, A.V., et al., Vascular dysfunction in glycogen storage disease type I. The Journal of pediatrics, 2009. 154(4): p. 588-591.

35. DiSant’Agnese, P.A., Andersen, D.H., and Mason, H.H., Glycogen storage disease of the heart: II. Critical review of the literature. Pediatrics, 1950. 6(4): p. 607-624.

36. Kishnani, P.S., et al., Pompe disease diagnosis and management guideline. Genetics in Medicine, 2006. 8(5): p. 267.

37. Kollberg, G., et al., Cardiomyopathy and exercise intolerance in muscle glycogen storage disease 0. New England Journal of Medicine, 2007. 357(15): p. 1507-1514.

38. Ozen, H., Glycogen storage diseases: new perspectives. World journal of gastroenterology: WJG, 2007. 13(18): p. 2541.

39. Shin, Y.S. Glycogen storage disease: clinical, biochemical, and molecular heterogeneity. in Seminars in pediatric neurology. 2006. Elsevier.

40. Verhalen, B., S. Arnold, and B.A. Minassian, Lafora Disease: A Review of Molecular Mechanisms and Pathology. Neuropediatrics, 2018.

41. Nitschke, F., et al., Lafora disease—from pathogenesis to treatment strategies. Nature Reviews Neurology, 2018: p. 1.

42. Gentry, M.S., et al., Lafora disease offers a unique window into neuronal glycogen metabolism. Journal of Biological Chemistry, 2018. 293(19): . 7117-7125.

43. Berkovic, S.F., N.K. So, and F. Andermann, Progressive myoclonus epilepsies: clinical and neurophysiological diagnosis. J Clin Neurophysiol, 1991. 8(3): p. 261-74.

44. Gentry, M.S., J.E. Dixon, and C.A. Worby, Lafora disease: insights into neurodegeneration from plant metabolism. Trends Biochem Sci, 2009. 34(12): p. 628-39.

45. Harriman, D.G., J.H. Millar, and A.C. Stevenson, Progressive familial myoclonic epilepsy in three families: its clinical features and pathological basis. Brain, 1955. 78(3): p. 325-49.

46. Hodskins, M.B. and P.I. Yakovlev, Anatomico-clinical observations on myclonus in epileptics and on related symptom complexes. American Journal of Psychiatry, 1930. 86: p. 827-848.

47. Minassian, B.A., Lafora’s disease: towards a clinical, pathologic, and molecular synthesis. Pediatr Neurol, 2001. 25(1): p. 21-9.

48. Pascual, S.I.P., Phenotype variations in early onset Pompe disease: diagnosis and treatment results withMyozyme®. Inherited Neuromuscular Diseases, 2009, Springer. p. 39-46.

49. Kishnani, P.S., et al., Early treatment with alglucosidase alfa prolongs long-term survival of infants with Pompe disease. Pediatric research, 2009. 66(3): p. 329.

50. Kishnani, P., et al., Safety and efficacy of VAL-1221, a novel fusion protein targeting cytoplasmic glycogen, in patients with late-onset Pompe disease. Molecular Genetics and Metabolism, 2019. 126(2): p. S85-S86.

51. Gertz, H.J., et al., Glycogen accumulation of the aging human brain. Mechanisms of Ageing and Development, 1985. 31(1): p. 25-35.

52. Sinadinos, C., et al., Neuronal glycogen synthesis contributes to physiological aging. Aging Cell, 2014. 13(5): p. 935-945.

53. Cavanagh, J.B., Corpora-amylacea and the family of polyglucosan diseases. Brain Research Reviews, 1999. 29(2): p. 265-295.

54. Gertz, H.-J., et al., Glycogen accumulation of the aging human brain. Mechanisms of ageing and development, 1985. 31(1): p. 25-35.

55. Moore, S.A., et al., Glycogen accumulation in tibial nerves of experimentally diabetic and aging control rats. Journal of the neurological sciences, 1981. 52(2-3): p. 289-303.

56. Mann, D.M.A., et al., Glycogen accumulations in the cerebral cortex in Alzheimer’s disease. Acta Neuropathologica, 1987. 73(2): p. 181-184.

57. Hetz, C. and B. Mollereau, Disturbance of endoplasmic reticulum proteostasis in neurodegenerative diseases. Nat Rev Neurosci, 2014. 15(4): p. 233-49.

58. Ronnett, G.V., et al., AMPK in the brain: its roles in energy balance and neuroprotection. Journal of neurochemistry, 2009. 109 Suppl 1(Suppl 1): p. 17-23.

59. Ronnett, G.V. and S. Aja, AMP-activatedprotein kinase in the brain. International Journal Of Obesity, 2008. 32: p. S42.

60. McGee, S.L., et al., AMP-activated protein kinase regulates GLUT4 transcription by phosphorylating histone deacetylase 5. Diabetes, 2008. 57(4): p. 860-867.

61. Cokorinos, E.C., et al., Activation of skeletal muscle AMPK promotes glucose disposal and glucose lowering in non-human primates and mice. Cell metabolism, 2017. 25(5): p. 1147-1159. e10.

62. Kishton, R.J., et al., AMPK Is Essential to Balance Glycolysis and Mitochondrial Metabolism to Control T-ALL Cell Stress and Survival. Cell Metab, 2016. 23(4): p. 649-62.

63. Lee, W.J., et al., AMPK activation increases fatty acid oxidation in skeletal muscle by activating PPARa andPGC-1. Biochemical and biophysical research communications, 2006. 340(1): p. 291-295.

64. Mobbs, Jesse I., et al., Determinants of oligosaccharide specificity of the carbohydrate-binding modules of AMP-activatedprotein kinase. Biochemical Journal, 2015. 468(2): p. 245.

65. Li, X., et al., Structural basis of AMPK regulation by adenine nucleotides and glycogen. Cell Research, 2014. 25: p. 50.

66. Koay, A., et al., AMPK β subunits display isoform specific affinities for carbohydrates. FEBS Letters, 2010. 584(15): p. 3499-3503.

67. DePaoli-Roach, A.A., et al., Glycogen Phosphomonoester Distribution in Mouse Models of the Progressive Myoclonic Epilepsy, Lafora Disease. The Journal of Biological Chemistry, 2015. 290(2): p. 841-850.

68. Bertoft, E., Understanding Starch Structure: Recent Progress. Agronomy, 2017. 7(3).

69. McBride, A., et al., The Glycogen-Binding Domain on the AMPK β Subunit Allows the Kinase to Act as a Glycogen Sensor. Cell Metabolism, 2009. 9(1): p. 23-34.

70. Vilchez, D., et al., Mechanism suppressing glycogen synthesis in neurons and its demise in progressive myoclonus epilepsy. Nature Neuroscience, 2007. 10: p. 1407.

71. Kakhlon, O., et al., Guaiacol as a drug candidate for treating adult polyglucosan body disease. JCI Insight, 2018. 3(17).

72. Pederson, B.A., et al., Inhibiting glycogen synthesis prevents Lafora disease in a mouse model. Annals of neurology, 2013. 74(2): p. 297-300.

73. Fan, T.W.M., et al., Stable isotope-resolved metabolomics and applications for drug development. Pharmacology & Therapeutics, 2012. 133(3): p. 366-391.

74. Lane, A.N., R.M. Higashi, and T.W.-M. Fan, Preclinical models for interrogating drug action in human cancers using Stable Isotope Resolved Metabolomics (SIRM). Metabolomics, 2016. 12(7): p. 1-15.

75. Fan, T.W.-M., et al., Stable isotope resolved metabolomics analysis of ribonucleotide and RNA metabolism in human lung cancer cells. Metabolomics, 2012. 8(3): p. 517-527.

76. Higashi, R.M., et al., Stable Isotope-Labeled Tracers for Metabolic Pathway Elucidation by GC-MS and FT-MS, in Mass Spectrometry in Metabolomics: Methods and Protocols, D. Raftery, Editor. 2014, Springer New York: New York, NY. p. 147-167.

77. Roma-Mateo, C., P. Sanz, and M.S. Gentry, Deciphering the role of malin in the lafora progressive myoclonus epilepsy. IUBMB Life, 2012. 64(10): p. 801-8.

78. Gentry, M.S., C. Roma-Mateo, and P. Sanz, Laforin, a protein with many faces: glucan phosphatase, adapter protein, et alii. Febs J, 2013. 280(2): p. 525-37.

79. Mercier, C. and W.J. Whelan, The fine structure of glycogen from type IV glycogen-storage disease. European journal of biochemistry, 1970. 16(3): p. 579-583.

80. Nitschke, F., et al., Abnormal glycogen chain length pattern, not hyperphosphorylation, is critical in Lafora disease. EMBO Molecular Medicine, 2017. 9(7): p. 906.

81. Raththagala, M., et al., Structural Mechanism of laforin Function in Glycogen Dephosphorylation and Lafora Disease. Molecular Cell, 2015. 57(2): p. 261-272.

82. Young, L.E., et al., Gas-chromatography coupled mass spectrometry for rapid and sensitive quantitation of glucose and glucose phosphates derived from glycogen. bioRxiv, 2019: p. 645556.

83. Sullivan, P.M., et al., Targeted replacement of the mouse apolipoprotein E gene with the common human APOE3 allele enhances diet-induced hypercholesterolemia and atherosclerosis. Journal of Biological Chemistry, 1997. 272(29): p. 17972-17980.

84. Sullivan, P.M., et al., Type IIIhyperlipoproteinemia and spontaneous atherosclerosis in mice resulting from gene replacement of mouse Apoe with human Apoe * 2. The Journal of clinical investigation, 1998. 102(1): p. 130-135.

85. Ros, S., et al., Control of Liver Glycogen Synthase Activity and Intracellular Distribution by Phosphorylation. Journal of Biological Chemistry, 2009. 284(10): p. 6370-6378.

86. Johnson, L.N., Glycogen phosphorylase: control by phosphorylation and allosteric effectors. The FASEB Journal, 1992. 6(6): p. 2274-82.

87. Soderling, T.R., et al., Phosphorylation and inactivation of glycogen synthase by phosphorylase kinase. Proceedings of the National Academy of Sciences of the United States of America, 1979. 76(6): p. 2536-2540.

88. Jensen, J. and Y.-C. Lai, Regulation of muscle glycogen synthase phosphorylation and kinetic properties by insulin, exercise, adrenaline and role in insulin resistance. Archives of physiology and biochemistry, 2009. 115(1): p. 13-21.

89. Thompson, J.A. and G.M. Carlson, The regulatory a and β subunits of phosphorylase kinase directly interact with its substrate, glycogen phosphorylase. Biochemical and biophysical research communications, 2017. 482(2): p. 221-225.

90. Printen, J.A., M.J. Brady, and A.R. Saltiel, PTG, a protein phosphatase 1-bindingprotein with a role in glycogen metabolism. Science, 1997. 275(5305): p. 1475-1478.

91. Magnusson, I., et al., Liver glycogen turnover in fed and fasted humans. American Journal of Physiology-Endocrinology And Metabolism, 1994. 266(5): p. E796-E803.

92. Petersen, K.F., et al., Mechanism by which glucose and insulin inhibit net hepatic glycogenolysis in humans. The Journal of clinical investigation, 1998. 101(6): p. 1203-1209.

93. Waagepetersen, H.S., et al., A Possible Role of Alanine for Ammonia Transfer Between Astrocytes and Glutamatergic Neurons. Journal of Neurochemistry, 2000. 75(2): p. 471-479.

94. Waagepetersen, H.S., et al., Demonstration of pyruvate recycling in primary cultures of neocortical astrocytes but not in neurons. Neurochemical research, 2002. 27(11): p. 1431-1437.

95. Yao, Y., M.J. Guiltinan, and D.B. Thompson, High-performance size-exclusion chromatography (HPSEC) and fluorophore-assisted carbohydrate electrophoresis (FACE) to describe the chain-length distribution of debranched starch. Carbohydrate research, 2005. 340(4): p. 701-710.

96. Cataldo, A.M. and R.D. Broadwell, Cytochemical identification of cerebral glycogen and glucose-6-phosphatase activity under normal and experimental conditions: I. Neurons and glia. Journal of Electron Microscopy Technique, 1986. 3(4): p. 413-437.

97. Brown, A.M., Brain glycogen re-awakened. Journal of neurochemistry, 2004. 89(3): p. 537-552.

98. Saez, I., et al., Neurons have an active glycogen metabolism that contributes to tolerance to hypoxia. Journal of Cerebral Blood Flow & Metabolism, 2014. 34(6): p. 945-955.

99. Luo, Z., M. Zang, and W. Guo, AMPK as a metabolic tumor suppressor: control of metabolism and cell growth. Future oncology, 2010. 6(3): p. 457-470.

100. Shaw, R.J., et al., The tumor suppressor LKB1 kinase directly activates AMP-activated kinase and regulates apoptosis in response to energy stress. Proceedings of the National Academy of Sciences, 2004. 101(10): p. 3329-3335.

101. Green, A.S., et al., The LKB1/AMPK signaling pathway has tumor suppressor activity in acute myeloid leukemia through the repression of mTOR-dependent oncogenic mRNA translation. Blood, 2010: p. blood-2010-02-269837.

102. Wei, Z., et al., Amyloid β protein aggravates neuronal senescence and cognitive deficits in 5XFAD mouse model of Alzheimer’s disease. Chinese medical journal, 2016. 129(15): p. 1835.

103. Schneider, F., et al., Behavioral and EEG changes in male 5xFAD mice. Physiology & behavior, 2014. 135: p. 25-33.

104. Russell III, R.R., et al., Translocation of myocardial GLUT-4 and increased glucose uptake through activation of AMPK by AICAR. American Journal of Physiology-Heart and Circulatory Physiology, 1999. 277(2): p. H643-H649.

105. Marsin, A.S., et al., Phosphorylation and activation of heart PFK-2 by AMPK has a role in the stimulation of glycolysis during ischaemia. Current Biology, 2000. 10(20): p. 1247-1255.

106. Fediuc, S., M.P. Gaidhu, and R.B. Ceddia, Regulation of AMP-activatedprotein kinase and acetyl-CoA carboxylase phosphorylation by palmitate in skeletal muscle cells. Journal of Lipid Research, 2006. 47(2): p. 412-420.

107. Ronnett, G.V., et al., AMPK in the brain: its roles in energy balance and neuroprotection. Journal of neurochemistry, 2009. 109: p. 17-23.

108. Faubert, B., et al., AMPK Is a Negative Regulator of the Warburg Effect and Suppresses Tumor Growth In Vivo. Cell Metabolism, 2013. 17(1): p. 113-124.

109. Oligschlaeger, Y., et al., The Recruitment of AMP-activated Protein Kinase to Glycogen Is Regulated by Autophosphorylation. Journal of Biological Chemistry, 2015. 290(18): p. 11715-11728.

110. Maragakis, N.J. and J.D. Rothstein, Mechanisms of disease: astrocytes in neurodegenerative disease. Nature Reviews Neurology, 2006. 2(12): p. 679.

111. Verkhratsky, A., et al., Astrocytes in Alzheimer’s disease. Neurotherapeutics, 2010. 7(4): p. 399-412.

112. Thon, V.J., M. Khalil, and J.F. Cannon, Isolation of human glycogen branching enzyme cDNAs by screening complementation in yeast. Journal of Biological Chemistry, 1993. 268(10): p. 7509-7513.

113. Fyfe, J.C., et al., Glycogen storage disease type IV: inherited deficiency of branching enzyme activity in cats. Pediatric research, 1992. 32(6): p. 719.

114. Valberg, S.J., et al., Glycogen branching enzyme deficiency in quarter horse foals. Journal of veterinary internal medicine, 2001. 15(6): p. 572-580.

115. Sun, R.C., et al., Noninvasive liquid diet delivery of stable isotopes into mouse models for deep metabolic network tracing. Nature Communications, 2017. 8(1): p. 1646.

116. Fan, T., et al., Stable isotope resolved metabolomics of lung cancer in a SCID mouse model. Metabolomics, 2011. 7(2): p. 257-269.

117. Fan, T.W.-M. and A.N. Lane, Structure-based profiling of metabolites and Isotopomers by NMR. Progress in NMR Spectroscopy, 2008. 52: p. 69-117.

118. Le, A., et al., Glucose-Independent Glutamine Metabolism via TCA Cycling for Proliferation and Survival in B Cells. Cell metabolism, 2012. 15(1): p. 110-21.

119. Lorkiewicz, P., et al., High information throughput analysis of nucleotides and their isotopically enriched isotopologues by direct-infusion FTICR-MS. Metabolomics : Official journal of the Metabolomic Society, 2012. 8(5): p. 930-939.

120. Gori, S.S., et al., Profiling thiol metabolites and quantification of cellular glutathione using FT-ICR-MS spectrometry. Anal Bioanal Chem, 2014. 406(18): p. 4371-9.

121. Sellers, K., et al., Pyruvate carboxylase is critical for non-small-cell lung cancer proliferation. The Journal of Clinical Investigation, 2015. 125(2): p. 687-698.

122. Fan, T.W. and A.N. Lane, Applications of NMR spectroscopy to systems biochemistry. Prog Nucl Magn Reson Spectrosc, 2016. 92-93: p. 18-53.

123. Fan, T.W., et al., Distinctly perturbed metabolic networks underlie differential tumor tissue damages induced by immune modulator beta-glucan in a two-case ex vivo non-small-cell lung cancer study. Cold Spring Harb Mol Case Stud, 2016. 2(4): p. a000893.

124. Turnbull, J., et al., PTGprotein depletion rescues malin-deficient Lafora disease in mouse. Annals of neurology, 2014. 75(3): p. 442-446.

125. Van der Ploeg, A.T., et al., A randomized study of alglucosidase alfa in late-onset Pompe’s disease. New England Journal of Medicine, 2010. 362(15): p. 1396-1406.

126. Chan, E., et al., Progressive myoclonus epilepsy with polyglucosans (Lafora disease): evidence for a third locus. Neurology, 2004. 63(3): p. 565-567.

127. Duran, J. and J.J. Guinovart, Brain glycogen in health and disease. Molecular Aspects of Medicine, 2015. 46: p. 70-77.

128. Beutler, E., et al., Enzyme replacement therapy for Gaucher disease. Blood, 1991. 78(5): p. 1183-1189.

129. Jutta, K. and P.G. Lankisch, Pancreatic enzyme replacement therapy. Current gastroenterology reports, 2001. 3(2): p. 101-108.

130. Brady, R.O. and R. Schiffmann, Enzyme-replacement therapy for metabolic storage disorders. The Lancet Neurology, 2004. 3(12): p. 752-756.

131. Melton, R.G. and R.F. Sherwood, Antibody-enzyme conjugates for cancer therapy. JNCI: Journal of the National Cancer Institute, 1996. 88(3-4): p. 153-165.

132. Bagshawe, K.D., Antibody-directed enzyme prodrug therapy: A review. Drug Development Research, 1995. 34(2): p. 220-230.

133. Amalfitano, A., et al., Recombinant human acid a-glucosidase enzyme therapy for infantile glycogen storage disease type II: results of a phase I/II clinical trial. Genetics in Medicine, 2001. 3(2): p. 132.

134. Sharma, S.K., et al., Galactosylated antibodies and antibody-enzyme conjugates in antibody-directed enzyme prodrug therapy. Cancer, 1994. 73(S3): p. 1114-1120.

135. Hansen, J.E., et al., Intranuclearprotein transduction through a nucleoside salvage pathway. Journal of Biological Chemistry, 2007. 282(29): p. 20790-20793.

136. Rattray, Z., et al., Re-engineering and evaluation of anti-DNA autoantibody 3E10 for therapeutic applications. Biochemical and biophysical research communications, 2018. 496(3): p. 858-864.

137. Mittal, S., et al., Interdependence of laforin and malin proteins for their stability and functions could underlie the molecular basis of locus heterogeneity in Lafora disease. Journal of Biosciences, 2015. 40(5): p. 863-871.

138. DePaoli-Roach, A.A., et al., Laforin and malin knockout mice have normal glucose disposal and insulin sensitivity. Human Molecular Genetics, 2012. 21(7): p. 1604-1610.

139. McI1wain, K.L., et al., The use of behavioral test batteries: Effects of training history. Physiology & Behavior, 2001. 73(5): p. 705-717.

140. Urbanc, B., et al., Neurotoxic effects of thioflavin S-positive amyloid deposits in transgenic mice and Alzheimer’s disease. Proceedings of the National Academy of Sciences, 2002. 99(22): p. 13990-13995.

141. Nakamura-Tsuruta, S., et al., Comparative analysis of carbohydrate-binding specificities of two antiglycogen monoclonal antibodies using ELISA and surface plasmon resonance. Carbohydrate Research, 2012. 350: p. 49-54.

142. Duran, J., et al., Glycogen accumulation underlies neurodegeneration and autophagy impairment in Lafora disease. Hum Mol Genet, 2014. 23(12): p. 3147-56.

143. Turnbull, J., et al., PTGprotein depletion rescues malin-deficient Lafora disease in mouse. Ann Neurol, 2014. 75(3): p. 442-6.

144. Head, E., A canine model of human aging and Alzheimer’s disease. Biochimica et Biophysica Acta (BBA)-Molecular Basis of Disease, 2013. 1832(9): p. 1384-1389.

145. Ahonen, S., et al. GysI antisense therapy rescues neuropathological bases of murine Lafora disease. bioRxiv preprint doi: https://doi.org/10.1101/2021.02.11.430846 (Feb. 11, 2021).

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

1. A method for treating a neurodegenerative disease, the method comprising administering, to a subject in need thereof, one or more compounds selected from the group consisting of a small molecule glycogen synthase (GYS) inhibitor, an antisense oligonucleotide targeting glycogen synthase, an antibody-enzyme fusion compound targeting polyglucosan bodies (PGBs), and combinations thereof.

2. The method of claim 1, wherein the small molecule GYS inhibitor is selected from the group consisting of: 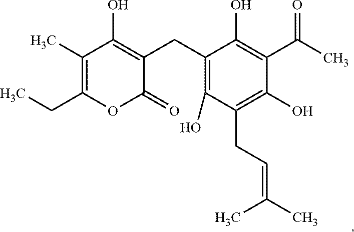 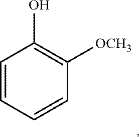 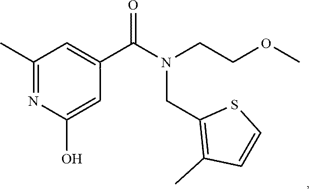 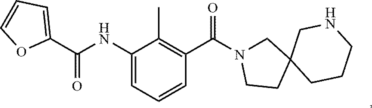 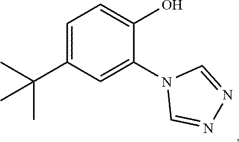 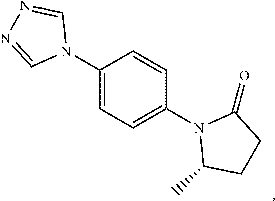
and a combination thereof.

3. The method of claim 1, wherein the antisense oligonucleotide comprises a nucleotide sequence complementary to an mRNA sequence coding for brain-expressed GYS.

4. The method of claim 1, wherein the antibody-enzyme fusion compound includes a cell-penetrating antibody comprising 3E10 linked to amylase or a variant thereof.

5. The method of claim 4, wherein the antibody-enzyme fusion compound is selected from the group consisting of VAL-0417, VAL-1221, and combinations thereof.

6. The method of claim 1, wherein the method comprises clearing PGB-like granules in the brain.

7. The method of claim 6, wherein the method comprises clearing PGB-like granules from cytoplasm in the brain.

8. The method of claim 6, wherein the one or more compounds comprise at least the antibody-enzyme fusion compound.

9. The method of claim 8, wherein the antibody-enzyme fusion compound comprises VAL-0417.

10. The method of claim 1, wherein the method comprises inhibiting PGB formation in the brain.

11. The method of claim 10, wherein the method comprises inhibiting PGB formation in cytoplasm in the brain.

12. The method of claim 11, wherein the one or more compounds comprise at least one of the small molecule GYS inhibitor and the antisense oligonucleotide.

13. The method of claim 1, wherein the method comprises:
first administering the antibody-enzyme fusion compound to clear existing PGB-like granules in the brain; and then
administering at least one of the small molecule inhibitor and the antisense oligonucleotide to inhibit new PGB formation in the brain.

14. The method of claim 1, wherein the neurodegenerative disease is selected from the group consisting of traumatic brain injury (TBI), Alzheimer’s Disease (AD), amyotrophic lateral sclerosis (ALS), epilepsy, and combination thereof.

15. The method of claim 14, wherein the neurodegenerative disease is Alzheimer’s Disease.


SEQUENCE LISTING (1 sequence)

SEQ ID NO: 1 - DNA, 32229 bp, Homo sapiens
ggcatggagctggacatgctctggcccctgctaatttcccagcatcccttgtaccaggcggccccagttctcaaccaccaggccttgcacatgctggcccctgagtccagactgctctccctgccctctcactgaattagatcaagtgctgccccctaggggaggccctttctgtccaacagccctgccaggctagttctaggacctctagacattcagacagatgaaaggcctccctgggagcccgatctccctgtgtgatctcacattcccgagggagatggatggatcaatattctctgctccagtggacatgagtcccatgaagggcacggcctgttctctcttgttcacctcgggactcaatgtctgtggtttgctaagtgatactctaaagtgctgagctcattcaacaagaatataccgaggcttcccatgttgacagttctccacagagaggagccagctaccaccccttctcacatgggggagggaggtgagtaaacaccacccgggacaattcaggacccccccagaggtccacacaaaacatggacacgagtgttcacggcagcactattcacaacagccaaaagatagaaacaacccacatgtccatcagctgatgaatggcaaaataaaacagggtctatttgtgcaaaggaatattattcagtcataaaaaggaatggagttcttttttcttttcttttttttttttttttgagacatagtctcactctgtcacctaggctggagtgcaatggcgcaatcttggctcactacaacctctgcttcctgggttcaagtgattcttctgcctcagcctcccgagtagctgggattacaggtgtccgccaccacacctggctaatatttttttgtattttttagtagagatggggtttcaccatgttggccaggctggtcttgaactcctgacctcaggtgatccacctgccttggcaccccaaagcgttggggttataggcgtgagccaccgcgcccggccaggaatggagttctgatacatgctacaacgtggatgaactttgaaaacattatactgactgaaagaagccacacacgaaagacacatatggtatgattccatctatatgaagtagccacaataggcaaatccatatacagagaaagattagtggctgcctagaactgaagatgggaggtatggaaggattagcggtgacaggtaagaggcatggggtttctttttggggtgacgacaatgttctaaaattgactgtggaaatggctgcacaagtctgtgaatatactagaaaacactgagctgtatactttaaatgggtgaattatgtagtacgtgaattctatctcaataaagctattaagaaaaaaaaaaaacaacaggctgggcgtagtggctcccacctgtaataccagcactttgggaggctgaagcaggcaaatcacttgaggtcaggggttcgagaccagcctggccaacatggtgaaaccctgtctctactaaaaatacaaaaattaggtcaggcacggtggctcacgcccgtaatcccagcactttgggaggtcaaggcaggtggatcacttgaggtcaggagttcgagactagcctggccaacatggtgaaacccagtctacactaaaaatacaaaaaattagccaggcgtggtggcaggtgcctgtaatcccaggtacttgggaggctgaggcaggagaattgcttgaacccaggaggcggaggttgtagcgagcagagatcgcgccactgcactccagcctgggcatcagagcaagactctatcactaacacacacacacacacacacacacacacacacacacacagatacaaaaattagctgggcgtggtgacgcgcacctgtagtcccagctactcaggaggctgaggcaggaggatcatttgaacatgggagagaggcagaggctgcagggagccaagatcacaccactgcactccagcctgggagacagagtgagactctgtcttgaaaaaaagaaaaaacaaaaaaacaatcctggatggtggatctttgcatgtctgttacattattttctgacactttggaatagttcataatttctaataaggaaaaggaagcctaagaagctagcatgctaaccgaggccacccatccccaggtctccttccttcccacaggtttgtgtttttttttttgagataagatctcattttgtcccccaggctggactgctgtggcctccacctcccaggctcaagcaatcctcccaccttagtctcccaagtagttgggactacaggtgtgtcccaccacacttggctaattttcttctcttttcttttcttttttttttttttttgagacacagccttactctgtcacccaggctggagtgcagtggcgcgatctcggctcactgcaaccttcgcctcccgggttcaagcaattctcctgcctcagcctccagagtagttgggactacaggcatgtgccaccacgcccggctaattttttttgtatttttaataaagacggggtttcaccatattagccaggctgctctcaaactcctgacctcatgacctgcccgcctcgaccccccaaagtgctgggattacaggtgtgagccaccgcacccggcctaattttcttatttttttgtagagacagggtttcgctatgttgccagggctagtcttaaattcctgggctcaagcaatccttctacctcggactcccaaagtgtgggattacaggcgtgagccactgtgcccagcccacaggtctttcttctcttcttcccctagagaggacatgcagttatccagcccagaggaagcctcctccctctggagccaatcgtttagactttctggccatcaatggtcacaagctttccctgtcactgtgtgaaccttcccacctaccgtctcctataggaagctggaacccaggagcatagaacattacccccagactacaagcagaagactaggcatccccaccccacaaaaaagacatgaatccaggaatcccaaatgtagctctctcaacataaacaggccccttaccaaggactgtctcctataaagactttctctttcatcataagtctctccaaaaccttgctgcccgctgttcccatgaaaaccaaggtcatttcaaaactcagcacacaccgtcccctcatacttccaatactttctccacggtagttctggtcatagccttcctcttctacttccctatccacctcctcctcctccttgccctggtgacagcaacagactcaaatctcatccccagctaccaacaactttctccccctcccttcctccatcttgactgggctcatcatggaaattgaggcacctgtaagcccagcccagtccctaagttcaggtcacattacctttttttttttggagacagagtctcgctttgtcgcccaggctggaatgcagtggcgcgatcttggctcactgcaacttctgcctcccaggttcaagcgattctcctgcctcagccttctgagtagctgggattacaggcctgcaccaccacacctggctaatttttgtatttttaatagagacggggtttcgcaatgttggccacgaactcctgacctgaagtgatccgcccgcctcggcctcccaaagtgctgggattacaggcatgagccaccacgccaggccaaaatcgcttctttattcaggtaattgagcaggtgccaggccaagtttcctggcctctccttcattcacatgtattcattcattcaaatattcaagtgataatttattgagcatttacactgagacgtagcccgtgtgaaatactggaaatatgaaaatggccaaaacggtagcatgacaagatcccgatttctgggtccctaatccttcagtcctctccactaagactctcttgttcctcgacaatttcttcccactaaaggctcactaggcgccctcgcgctctcagacacactgaacaggtacctgcctgtgcttccagcttggaactcaagtgccattggcaccactaacaatccaaggctcccaaatggctcgaagcgcaaggtcctacaaccctcctcatcacacactggcgccaaggactgcggaaccggccgttgcgccaagatgattttgaaagttggagttcctatagcatgatggggccagacccgagattctgggatcccagccccctccccgcctcagatccagaagtccagcccccatgtccccttctcccccagatctgggtggccgagcttcctcctctttcagacccaggagtctgaacccccagatcccccctccctcagacccaggagtcagggcctccagcatcctcctctctcaaactcgagactgctagctcgctcctcaccctccatgcacccacacttacaacggttgccatgatgctcaccaactgccagagtcctagcggaaacggaaacgcgcgaggagcgagatgttctggctcctctaggctagctggttcttcaaagatctcgatggttcctcataaatatgtaaatattgtgggggcgtggcgggactttatgccccgccctcccaccgaggctgcgctggaagacgggcgtccggaactcctgggtccattcccaatgtctcctggctcctcctccttttgctcagccccaccaccgccccgctcaggccgattgcaaaattcttagctgttctttgagtaaccacacgtcctccacgctcctttcctctttatcccaccctcctcctcctccccactttgaggcctccagctccagattgacagagctgaaggccactcgtttccgtaaagcgcagcggcaagctccgccccctggaggtctcacggccgaggcggggccgggacacagcgactccaacccgttccaccaatgaggacacgcggaaatccccgcccccgggcgtgggccaatggcgctggctggggggggcggccaccgcctcctggcggctgcgaggtttcactgcaggggcgccagtgggctcagtgacgctgcggcctccttctgcctaggtcccaacgcttcggggcaggggtgcggtcttgcaataggaagccgagcgtcttgcaagcttcccgtcgggcaccagctactcggccccgcaccctacctggtgcattccctagacacctccggggtccctacctggagatccccggagccccccttcctgcgccagccatgcctttaaaccgcactttgtccatgtcctcactgccaggactggaggactgggaggatgaattcgacctggagaacgcagtgctcttcgaagtggcctgggaggtggctaacaagggtgagcacgccaggcgtcggggcccttgacgagaaggacagtagtggatgggggcccggaactctgagttgtaggaggacggggctactaggggaacccttgtgacctgggcaaggaggctgtgtccttgaggaggggctgggaagccggaggtgggtgggagagggatggttcgtttgacttaagatgaaaggaggctggaagcccgggttctgggtcctagggaagagggcgctaaaatgagactcctaggtcctggaggcaggcttccgagagactagatacctgggtcctgaacagggaaggggatggggggatgggatttctgcgtgcaaacctgggaagctgccggggtttcagacctatgaccctaacagcatggtctaggggataaaactccagatttgagagagagctgactgggagataggaggcctgagcggcggtgggaggacctgaggatctggctgcttaagaataaagatgggacgggctggagactgtaatgaatgcctgggctttgaaggagctgagggcctgagctattagttcctaggagtagaggagcccatggcctgggacacatgcgtactggaataggggaaggctgggaagtcctaagttagtcagaggctgggccctaggatgggaggggctaggagcctgcatttctgtgtcttgggcttcagaaagctgaggttctggccttggagaaggctggggctgaaatccccaggaacaagccaatctcacacctcaaagagcagggtctggggggttgctttgctgggtcttggaggagaaagggactgtgtctcccaggaggagagggggcccgtgaacacccagccgggtatcgccagggcctgggctccaggatgggaggaggctgggggctggggctcctgggtctgggatagggtcatgggagaggcagagccctgcatgctggtgtccctagtcagggagaggaaattgggaggcaggtcagcaaggtccctgggggcagggaatgaccatgcaggcttctcttgggcaatggtgagaagctggattggccatgagggggtctccctcactgggttttggagcctggctgcgatttaaacccagctcctgtgagctgggaactcgggaacctgagtccgggtagtcagtcctcagctctgccatccttagagccccagtgcctaattccttcagaccaaggggtcccggcacccagccccttctctctcagacccagaagtctagcctgagccccctcctccttcagggtcctgaccccagcccccttctcccttagacccaggaggactgccctccagccctcgtgttcccctcatccaagcttccaacccctactccaaactatgggctcccagtcccctcttccccctgagctagaggtctggggcctgagtgccccctacatccccggcccaccccaccccaggccccagaactatggacctcggccggggcctgccctcacacgcttgggcccacagtgggtggcatctacacggtgctgcagacgaaggcgaaggtgacaggggacgaatggggcgacaactacttcctggtggggccgtacacggagcagggcgtgaggacccaggtggaactgctggaggcccccaccccggccctgaagaggacactggattccatgaacagcaagggctgcaaggtgggacgtggcccagcccagggcagaagcgggtggacagcctgccatcggggtgggagccacagacggagagggtgcagggtggacagccccagccagagagcgaggcaggcttggacacaggaaggaggcgtgataggacacccagacagatgcagggcctggaagtgggtgggtggatcccagacatgggcgaatggacagacagatggcaagtatgagacagactgagacggagatccaagaggccaaggtcctcagacccagagagaaagagagcaagatcgaaagacagcctggtcaacatggcgaaaccccgtctctaccaaaaatacaaaaattagctgggtgtggtggcgggtgcctgtaatcccagctactcgggaggctgaggcaggagaatcgcttgaacccgggaggcggaggttgcagtgagctgcgattgggccactgcactccagcctgggcgacggagcgagactctgtctcagaaaagaatgccagccagccaggcagagctggggagaagtgggcctgaggtcaggaaggagcaggattctggagcttgaagttcaagttcaccctccacttgctgcctgtgtgtccttggctaatggacatgctaatagcgtctaccttctagcggtgctgggaggaggaaacagttaataaggacaaagtggttagtagagttcttggcatgaaggtagcacattgtgtgggcggtgattgtcattgttatgatcatcgtcattattgttactaacagccaagcttggagagtgagggggacaggcagaccaagggagaagggtggatggagccacaggcaagagtaatagactggggacagaagagaggccaccagatgtttcagaaagccagtcctagaaggagagagaagcatggaatatcggggaagagaggagaggtcagtggacagacccacaggacagagtctgagatgggcagatcagggattagagaggagggggagttccagggccccagggtgaagacggaaagagacacgctgttgtccaaggagaaagccatcaggcggcacgctgttgtccaaggagaaagccatcaggcggcagggctaagggacagacaggtccaggctggggatggagaggcactgtggttcctttactcattcacagcctctggtttggggtggggtctggaatccattcaggtccgactgtgtccctaagggaacccagaacagcccagtcgcttcctgagggcagctgcttcccacgtgctgggggcctaagaatagcccccccccccccccagcaaaagggcagacaacagctgggagggggagggacctcagagcgaggctccaggcagcaaaaagccaggtcggcgaggagcggggctgcttctccactgctgtggcagcaggcccagaaccaaagccatggtctcagggaggaaaacagccaactggagaaatgtggcagaggcagaagtgagacagcaagtcttagaaacacggcagtggctagaaaaatgcagccctggggcacggacagaggatgggctatgttagaaatgtgtggcgtggccgagggcggtggctcatgcctggaatcccagcactttgggaggccgaggtgagaggatagcttgagcccaggagttcctgacccacctgggcaacatggcgaaactctgtctctactaaaaatacaaaaaattagccaggcatgatggtgcgtgctcgtgatcccagttactcaggaggctgaggtgggaggatgggatgtggaggctgtagtgagccataattttttttttttttttttcagatagaatctcactctgtcgctgagtctggagtgcagtggtacaatctcgactcactgcaacctccgcctcccgcgttcaagcaattctcctgcctcggcctcccaagtagctgggacctcaggcacatgctgccacgcccggctaattttttgtattttagtagagacagggtttcaccatgttgcccaggctggtctcaaactcctgagctcaggcaatccgcccgcctcggccccccaaagagctaggattacaggcgtgagccaccgcgcccagcccagtgagccatgattgcaccactgcactccagcctgggtgaccggagtgagaccctgtcaaaaacaaaaacaaacaaaaaaaaatggaagaaagaaagaaaaaggagagagggagggaaggagggagagagggaaggaaggaaggaaggaagcaaggaaggaaggaaggaggctgtggactacaaaatgggcctatggatagaggaaagaggtcgcaggtgacagatatgtgatgaaaggaacagaaacaaggcatcatggggaagaaaagtggcagcatgaacagtaagagaatcattacaggctgggtgcagtagctcacgcctgtaatcccagcactttggtaggctgaggccaggagtttgagaccagcctgggcaacatggtgaaaccctgtccttacaaaaaagttaaaaattagccgggatgtgataccttgtgcctgtggtcccagctacgtgggaagctgcggtggaaggattgcttgagcctgggagatcgaagcttcagtgaaccgtaattgcaccactcccttccaggctggaggacagagcaagaccccgtctctgaaaataaaaaagggcctgctttaggtggctcacacttctaatctcaacactttgggaggctaagcaagaaaactgcttgaacgcaggagttcacgatcagcctgggcaacatagtgagaccccatctccacaaaaaattaaaaaatcagcctggcatggtggcccacgcctgtaggaggtgaggtgggaggattgactgagccagggaggttgaggctatagtgaaccatgttcacaccactgcactccagcctgggcaacagagcgagaccctgtctgaaaacaaaaaagaagcatcccgtggaaggaattagcatcagaaagaaaagacagtgggagatgcagatggttggtaagagaaatgtggcagcaagtacagaaacaaggcagcaggtaggggaaatgatttgcagagtggccagacagggcagtgggggcatagacacaggacatgtgacatatgtggttataagcagagaaaggaggcagcgagtgggagaagggtagcagttacaggaattaaacaggaggctgggtgcggtggctcacgcctgtaatcccagcactttgggacgccgaggcgggcggatcacaaggtcgggaaatggagatcatcctggctaacacagtgaaacctcgtctctactgaaaatacaaaaaaaagtagccgggcgtggtggcgggccgctgtagtcccagctactcgggaggctgaggcaggagaatggtgtgaacccaggaggcagaggttgcagtgagccgagatcatgccacggcactccagcctgggcaacagagccagactccgtctcaagaaaaaaaaaaaattagccaggtgtgggagcgggtgcctgtaatcccagctacttgggaggctgaggcaggagaattgcttgaacccgggagacagaggttgcagtgagctgagatcgcaccactgcactccagcctgggccacagagtgagactccatctcaaaaaaaagaaagaaagaaagtaagcagggggcacggaaaatattgcaacagagatggaaaaaatgtgtagcaggaataaatcatgcaaatagagaagtaaggctaaggcagcaggtgagaaattagcagcagatatggaaacagaagataagagagctgaggcttaagtctatagacatctgcctgcaaatgggagaaccacggatgagtgaagatatgaggctgaagcccagagcctccctatggtgcctccccaaccccatcccaggtgtatttcgggcgctggctgatcgagggaggccctctggtggtgctcctggacgtgggtgcctcagcttgggccctggagcgctggaagggagagctctgggatacctgcaacatcggagtgccgtggtacgaccgcgaggccaacgacgctgtcctctttggctttctgaccacctggttcctgggtgaggtaggccccgtctgacacattccacccatacctgaaactacaatgaccatcagcccctgggccagaggatgggcttctccctgggcattctgggagtcatagggtcctttaatccaaggattagctctggcctttgaggacatgaacatccaagggcccaggctctactctcgatgccaacttcatttactctagtcttccaagcatagggattcttggaatcagcctcaaagtgaggctggcgggcttgcccgaggaattctaggagttgtagttctctagtgtgatattgctggactcagctctcagatgctgccctggatgctcatgaatccccagcctgacctggttttgacccccttctaggttttaagaggctcaagaagcttagtttccaggctgggcacggtggctcatgcctgtaatcccagcactttgggaggccgatgggggaagatcacgtgaggccaggagttcaagatggccaaacctcgtctttactaaacatacaaaaaaattagctgggtgtggtggtgggcacctgtaatcccaaatcccagctacttgggaggctgagacaggagaattgcttgaacctgggaggcgggaattgcagtgagctgagattgcatcacggcactccagcctgggccacagagtgagacactgtctcaaaaaaaaaaagaaaaagaaaaaaaaaaaaagaagcttaatttctcatgccctgggacaaataattgaggaactggctgctcctatggcattctgggagttgtagtccctttgccaatgttgggtgcagacagcgtacccttgagacaaggggtctttggaaactatgtccaggacctgggaacatgaaaacttcagtctgtccaatctaaccctgccaggacttagatggttggccaaaaaatcctcagttcctgtcaacccctgaggaggcagtcacttgagcccaggagttcaagaccagcctgggctgcctcagggatttacaggtccctgtccggttgggattgctgcagtgggccgctgcccactctgtgcagtagtggtgacagattgacctctccccgagtccttgtccctactggggattgccgattcttttcccaacactcgtggagacagtggccctgtccctgttgcccacagttcctggcacagagtgaggagaagccacatgtggttgctcacttccatgagtggttggcaggcgttggactctgcctgtgtcgtgcccggcgactgcctgtagcaaccatcttcaccacccatgccacgctgctggggcgctacctgtgtgccggtgccgtggacttctacaacaacctggagaacgtgagctgggaccgtggcagatgggggactgcgagtatgccaggaaaacccaaagctctggggggcccaaggggatggcaccccctcttctcccaggatccaggagtccaagtccccagacccctcctacctcagacccaggaatccagcccccagcaccaatttctcttgggaccccagtgtctggacctccagagtcttctccccaatccttctaaccggactgctgtcccctcccacccagttcaacgtggacaaggaagcaggggagaggcagatctaccaccgatactgcatggaaagggcggcagcccactgcgctcacgtcttcactactgtgtcccagatcaccgccatcgaggcacagcacttgctcaagaggaaaccaggtaggggctgggctgaagtccccagacgtgaatgaggtaggcagctgtctatgccaaagccagttgctcctcagcccaagaagcccactgtaagttgaaatagcataaaccgaaaacgtatttcggctgggcatggtggctcacacctgtaatctcagcactttgagaggccaaggcaggagaaccacttgagcccgggaatttgagaccagcctgggcaacatggcgaatccctgtctaaaatattcgccagacatggtgtgtgcctgtatccctagctactcaggaggctgaggtgggaggattgctcgagcctaggaggtcaagggtgcagtgagctgtgatcgtgccattgcatgatttgggtggcctggagagaccctgtcacagcctgggtgacacagtgagaccctgtctcaaaaaataaaaaaatgcatttaatacacctaacctactgaacatcatagcttaggctagtataccttaaatgtgcccagaacactttatgttagcctacgactgggcaaaatcatccaatacaaatctgttttattttatttttatttatttatttatttttgagacggagtctcgctctgtcgcccaggctggagtgcagtggcgctatcttggctcactgtaagctccgcctcccaggttcatgccattctcctgcctcagcctcccaagtagctgggactatacgcgcctgccaccacgcctggctaattttaatacaaacctattttataataaagtgttgaataggctgggcgcagtggctcacacctgtaatcccaacactttgggaggccaaggcgggcagatcacgaggtcaggagatcgagaccagcctggccaacatggcgaaactccatctctactaaaaatacaaaaattagccgggcgtggtggtgggtgcctgtaatcccagctactcgggaggctgaggcaggagaatcacttgaacccaggaggcggaggttgcagtgggccgagatcacaccactgcactccagcctggacaacagagcgagactccatctcaaaaaattaaaaaaaaaaagtgttgaatatctcatcaaaatccaaaattggactcatgtggatgtatgtaactttcacaccatcataaggttgaaaaatcataacttggctgggcacggtggcttacacctgtaatcccagcactttggaaggctgaggtgggcagatcacctgaggtcatgtgctcaagaccagcctggccaacattgtgagaccccctctctgccaaaaatacaaaaattaggtgggcatggtggcaggcgcctgtaatcccagccacccagctactcgggaggctgaggcaggagaatctcttgaacccaggaggcagaggttgtcgtgagccaagattgtgccattgcactccagcctgggcaacaagagcgaaactctgtctcgaaaagaaaaatcataacttggaccatcctaagtcctaagctggggaccatcagttaccatttttttacctaaagaatagagaactacagctcccagaaggctgtgggggtggtgacctgtatttgaggctctctgactatatactctggaggctaatgggaggtataatttctttcatattaatttcagatatgataaaattctgtaattggctaatgtagattggtcttatgaaagggaatggaagatcagtggactttgcttttgagaaaggttaggtcagaactgggcattcttcccagcccttagtatccgttctcagtgagtataaggtgagaactacaactcccagaaggcctttgactggttagctctatatgttccagaactttgccctaggcgctgatgggggttgtagtttttcgttgatatactttaatttctatgaggagaagaaatgattgggtccctgtaagaagcttggaagtagaggatatatcctaggagaagcaggactttggggatgtttaatcatttccatctcagggtgggctaagaggaaacaaaagaagtacaactttcccagaatcaccagtgtggatcagatccctgttagccatttgcagtagctgaggagacttatacttgtgcttgtgaacctaagcaagtaatattaggcaagaaggcatcttggagtcagggtaggtgtgtcaccaagggaactggccaggcatggtggctcacacctgtaatcccagcactttaggaggccagggcaggaagactgcttgaagccaggagtttgagatcagcttgggtaacatgacaagaccccatcactacaaaagtaaaataaataaattagccgggtgtggtagtgcacgcctgtagtgccagctactcgggaggctgaggagggaggattgcttgagcccaggaggtcagggatgcaatgagctgtgattgtgccactgctctccagcctgagcgacagagagaaaccctgtctccaaaaacaaacaaacaaacaaaaaaggtgggaggggggacctcataggaacgctcttgaaacaaaaaaattgcaaaaggcaaagggtgttttcttcatttattcattcaaccacaacattgatctgaatgaacaaataaaacatgggaccctcacccccaatcccagatattgtgacccccaatgggctgaatgtgaagaagttttctgccatgcatgagttccagaacctccatgctcagagcaaggctcgaatccaggagtttgtgcggggccatttttatgggtacgtggggcatatacctaggtcttaagagaggagggcgttgggagcctaaccatctaggtctgggggagggggcagctgggggcccagactcctgggtctgagggagggggcacctgggtattcggcctccttaattcttgagcgctggaacaatgggctcaggattaaaggaaatctccatctaaagagaagaactacaactcccaggaggccccgtgatgcctctgtattacctaagacatcctctgcccccccacccatagcttctgggatttgtagttttgaggccagggctagcgggtgagggctttgggctttaccgtgccttgtgggttctttaggcatctggacttcaacttggacaagaccttatacttctttatcgccggccgctatgagttctccaacaagggtgctgacgtcttcctggaggcattggctcggctcaactatctgctcagagtgaggcctgggctatgaggggacagggagggcaagcaaagcagtttataactagggagaacagccaggcacagtggctcacacctgtaatcccaacacttttggaggccaaggcgggaggatcacttgagcccaggagatggagaccagcctgggcaacatggtgaaaccccatctctacaaaaaatacaaaaattagccaggcatggtggctcacacgtgtagtcccagctactcgggagggtgaacggggaggatcacgcgagcccgggaggtggaggttgcagtgagccaagatcatatcactgcactccagcctgggcgacagaaaaagacgctgtctcaaaaacaaaacaaaacaaaacaaaacaaaaaattacagaacagaaacccaagaagccaggaagatgggggtaggaaagcccctgagtatggtggggggccgtgaggggagtatagagacccagaaagaaggtcagagtccaggtggggactgtcctataattctccttgacaggaatgggtatggcaggatcccagtggtcttgacagaaaggctggctggttccaggcttcccccacatgatcctggcctccccctcctttctttctgcgcaggtgaacggcagcgagcagacagtggttgccttcttcatcatgccagcgcggaccaacaatttcaacgtggaaaccctcaaaggccaagctgtgcgcaaacagctttggttagcagccctcgctccggcccctggcgcagcccactcccagatgcatgctttgggtcctgtgtttctcctagagtctcagttttttgttttgtttgtttgtttgtttgtttgtttttgagacagtctcactctgtcgcccaggctggagtgtagtggtgcgatcttggctcactgcaacctccacctcctgggttcaagcaagtcatctgcctcagcctctcgagtagctgggactacaggtgcacaccaccatgcctggctaatttttgtattttcagtagagatggggtttcaccacattggccagactagtctcaaactcctgatctcgtgatctgcccgcctcagcctcccaaagtgctggaattacaggcatgagccaccgcacccggccctcttttttttttttattttgagatggagttttgctctgtcgcccaagctggagtgcaatggcatgatctcggctcactgcaacctctgcctcctgggttcaagcgattttcctgcctcagcctctgagtagctgggactacaggggtgtgccaccacacctggctaactttttatttttattagagacaggttttcactatattggccaggctggtcttgaactcctgacctcaggtgttccgcctgcctcagcctcccaaagtgctgggattccaggcgtgagccaccacacctagcctttttttgtatttttagtagagatggggtttcaccatgttggccaggctgatcttgaacacctgacctcaggtgatccagccgccttggcatcccaaagtgctgggattacaggggtgagccactgcgcctggcttagtttctttttcttttctctgatttttttttttttttgaaacagagtctcactgttgcccaggctggagtgcagtggcgtaatctcagctcactgtaacctccgcctcccagcaaagctcagtttcttcatctgtaaaaccgttcattcattagcagagatactcactgagcatctactatgtgctagctaatttttatatttttagcaccgatgggatttcaccacgttgaccaggctggtcttgaactcctgacctcaagtgacctgccctcctcatgtattccaggtcctgggaatacagctgtgaaccagaaagataagatccctttcctcatggagcttgtattctagaaaatcctgtagacggaatttacttttgggcaaaaataaagcagaactggaatatgagacggggttgtcaggacaggccacactgagaaagtgttattgggcagagacgaggaaaggtgacgagggagcaagccctgtgggtatctgaggaacagcaaactcgaaggccccgaggcatgattgagacgcggtggggagtcccatggggctggagtgacatgtctcatagagaaggggtagaaagtgaggcaagatggagggcacctgtagtgccagctacaggaggcaggaggatcattttggccctggaggttgaggctgtagtgaggcatgattgcgccactgcactccagcctaggcaacagagtgagactctgtctcaaaaaaataaaaaagaagtcgggcgtggtggctcacacctgtaatcccggcactttgggaggccgaggcgggtggatcacctgaggtcgggagtttgagaccagcttgaccaacatggtgaaaccccgtctttcctaaaaataaaaaaaatcagcagggcgtggtgacacacatctgtagtcctagctactcgagaggctaagcaggagaatcgctcgaacccgggaggcagagattgcagtgagccaagatcgcgccaatgcactccagcatgggtgacagtgagactcggtctcaaaaaaaaaaaaaaaaaaaagaaagaaagaggctgggcgcggtggctcacacctgtaatcccagcactttgggaggctgaggagggcagatcacttgaggtcaggagttcgagatcagcctggtcaacgtggtgaaatcccatcggtactaaaaatataaaaattagccagatgtggtggcgcacgcttgtaattgcagctactcaggaggctgaggcaggagaatttcttgaacctagaaagtggccgagattgcgccatcctgggcgacagagcaagactctgtctcaaaaaaaaaaaaaaaaaaaaaaaaaaaaaaaaaaaaaaaaagaaaagaaaaaagaaaaagagacaaagaaagaaaaataaatgaggccagagctatgggaggattttaagtggaggagtgaccgtggggctgttgtgtgacactgctagctttacttgtgtgcctaagtggtcctttctcagaatcttactttcttttcgtataaaatggggcttagcattgccatctgcaggacaagtacaagtcctaataatagctgacatttctggaaggcttgccatgtgtactttttggcatgacatcattgggtcctcatcgcaaacctctgaggttgattctattttcatcctcctctgaaaaatgagtcactgcagtcagagagggaaggccacctgtccctgaccacacagctgatgtcaggcacgtgggtcctaagccttccagggcctgccccctgggtccccatcctccagcctggaggccccagggcacggcttccctgctcacacctcagtttccttgctatcctagaaacctaagggtagctgggcgcggcagctcatgcctgtaattccagcactttgggaggccgaggtggccaattaccagccagaggtcaggagttcgagaccagcctggccaacgtggtgaaaccccgtctctactaaagttacaaaaattagccgggcatggtggcaggtgcctgtaatcccggctactcaggaggctgaggcaggagaattgcttgaacctgagaggcagaggttgcagtgagccaagatcgcaccactgcgctccagcctggacgacagagtaactctgtcaataataataataataataataaataaactgctgggcacagtggctcaagcctctaatcccagtactttgggaggccgaggtgggcagattacctgaggttgggagtttgagaccagcctgaccaacatggagaaaccccgtctctactaaaaatagaaaattagccgggcgcggtggctcatgcctgtaatcccagctactcgggaggctgaggcaggagaattgcttgaacctgggaggcagaggttgcagtgagccgagatcacgccattgcattccagcctgggcaacaagagccaaactccgtctcaaaataaacaaacaaactaactaaagaagcctaacagtaaatggcagctggtgtgtatgtgaccctgttgctctgcttcctccagggacacggccaacacggtgaaggaaaagttcgggaggaagctttatgaatccttactggtgtgagtgccccaccctcagccctgtgccctcctaggccctgactaacccctctcattactccgttctcacatgcatgcccctgcttgccttgcagtgggagccttcccgacatgaacaagatgctggataaggaagacttcactatgatgaagagagccatctttgcaacgcaggtatggattggacctctgtgcagagagatagcagttcctggcagccccagaggcagcttgccaggccagctcagaccccagagattgctgggagtcctgctttctcctgcagccctttatgaagggatctattctgaggtccacagaagggcacgtgctgactcgtgcaacttcagttgctggaggcgaggcacttagggtgggctgttttctgtggcttattctgccacgagggttgctgggacgtgtggtttctctgggattctggggtcaatgagcaaaaggaagcttatttgtttatttatttttattatatttatttatttattttgagatggagtctcgctctgtcgcccaggctggagtgcagtggcacgatctcggctcactgcaacctctgcctcctgggttcaagcaatactcctgccttggcctcccaagtacctgggattacaggcacatgccaccacacccagctaatttatatatttttagtagaaacgagatttcaccatgttggccaggccagtctcaagctcctgacctcaagtgttctgtctgcctcagcctcccaaagtgctgagtttacaagcgtgagtccctgttcccagcccaaagggaagtttaaaaggaagtgtagaaatgtaataaagggccaggtgtggtggctcatgcctgtaatctcagcactttgagaggccaaagcaggaagatcacttgagaccaggaggctgaggctgcagtgagccatgactgtgccactgtacaccagcctgggggacagagccagatcccacactagccccccacccacccccaaaagaaagtcaaggcccagcttggtagctcacacctgtcagtattcccagcactttgggaggctgaggtgggaggatcgcttgagcccaggagtttgagaccagcctgggcaacatgatgaaaccctgtctctaccaaaaatacaaaaatgagctgggcatagtggcatacacctgttatcccagctacctgggaggctgaggccagaggatgccttgagctcaggaggtcaaggctgcagtgagctatgattatgccgctgcagtgagctatgattatgccactgcactccagactgggcaacagagcgaggcggacacatacacacacacacgaaagaaggaaaagaaagaaaagaaaaggtcaagacagccataaaagaattacaactctcagctctccttgggccttgctgcacacggctccacctctacccctaaaactgctgggagtggtagttttatcgggccactttagggacgcagtcaggaggtgagcaaaagcacccagcattctccagaaaacattgcaaactctcagcagctgcggtttcttatttccagaggctgtgtgttggtccccagacacccctgggaattgtagctgctttagatccctaacttcagagagaattggcatttataaaatgagaacttaccgtcattctattgagtctaggggcccaaaaaactacaactcccagcagcccctgctggctttctggggcattgacgcttcccttgtccagaggcttctgggagttagagttctttcattctctggggtccagggacttcagtacttgtggtctacattggggagccatttcctggggaatggaaacccagacagctaagttgtagaaacaaaacgtgaactgcttttggctaacctataatttccaatagtccctaaagcagcccacctcattatgaatgtttctgccatcgggatgctgggagttgtagtttctcgggcccctagactcagtagagtggtggtaagtaaaatctttaatgagctatgatggaacagttgctaagaactatacaccccagcagtcctcaggacttcctatcacactagctctgtcttgctccagagactcttggaaattgtggtttctttgggcctttgttagggttggaggtgaattttcttttttcttcttttttttttttttttttttttttgagacggagtctcgctctgtcgcccaggctggagtgcagtggcgcgatcttggctcacttcaacctccgcctcccggattcatgccgttctcctgccccagcctcctgaggcacccaccaccacgcctggctaattttttgtatttttagtagagacggggtttcaccgtgttagccaggatggtctcgatctcctgacctcgtcatcctcctgcctcggcctcccagagtgctgggattacaggcgtgagccaccacgcctggtggttggaagtgaattttcataaggaaactacaactcccagcagtctccaaagcttgctgccatattggcacagtctccacccccattgactggggatagttatatttcctttgggtttctttaataatagagcccagagtacagaacaaggtagctgcaactcctcatagtccttggaggtaagggagttgctaacacggctttgcctcaaagacagctgggaattctagttctttgaagctcatggtggttaagggaaggaaaacatttccaggccaggtgtggtggctcatgtctgtaatcccagcacttcgggaggccaaaacggatgcatcgcttgagcccatgagttcgagaccaacttggccaacatagtgaaaccccatctctattaaaaaaaaaaaaaaaaaaaattaggctgagcgcaatggctcacgcctgtaatcccagcactttgggaggccaaggcaggtggatcacgaggtcaggagatctagaccatcctgatttaacacagtgaaacctcgtctctactaaaaatacaaaaaattaaccgggcgtggtggtaggcgcctgtagttccagctactcgggaggctgaggcaggagaatggcatgatcctgggaggtggagcttgcagtgagccgagattgtgccactgcattccagcctgggcgacagagcgagactccgcctcaaaaataaataataataatttaaaagaaaagaaagaaaacctttacaggttgtactagtgggaatccacaactcctggtgatcacttgggtttgttgccccacgtccggtgcatttgtggtctccttggctcccccagggtccgcatggctcatgaggaagtagggagttctctgtcccttccatgaccccttacccttcttgtggctcctgcagcggcagtctttcccccctgtgtgcacccacaatatgctggatgactcctcagaccccatcctgaccaccatccgccgaatcggcctcttcaatagcagtgccgacagggtgaaggtgagggcatttggtcagggctgggttgggcacggcagagggaaggggttgggtcctgggcctcacacactccattcccctataagttttatcaaaggtattgctgtggttagaaagaaccctggcttctgaggcaggcagacctgggtttgaacattggtcctgccaaacacatgctgtgtgatgctagacaagctttttaccctctctgggccaccttgggatctcagggcttagctaaataaggcacttgggctgagtccttctttttcactaccaggtgattttccacccggagttcctctcctccacaagccccctgctccctgtggactatgaggagtttgtccgtggctgtcaccttggagtcttcccctcctactatgagccttggggctacacaccgggtgagtgtagtgggcaggggacagcgtggtcatggcatcctagtcagagctagcatgtcttggggcgaggcactggccttctctgagcctgttgccttctttgcaaaacaggaacaatggtaatcacctgttctgctacagcgcgtgcttttgtgatacaatagctcatgtattgagaaactagaagactgggttttgaggactagtgaactggatgtcacagttcacactgctttctcccagaaagcagtggccccccaaatagtggcaatgaaatgcacaattcacaaatacagccgagtgtggccaagtctcctcgtaagacgttcccagtgcccatcaactccaccttcctcttgttggttcacattagccagtgtttttaaagctaaataaggctgggcgtggtggctcatgcctgtaatcccagcacttggggaggccaagatgggcagatcatgaggtcaggagtttgagaccagcctggccaatatagtgaaaccctgtctctactaaaaatacaaaaattagccaggtgtggtggtacgcacctgtagtctcagctacttgggaggctgaggcagagaattgcttgaacttgggaggcggaggttgcagtgagctgagatcatgccactgcactccggcagtgctgtgctctgggtgacagagcgagactccgtctcaaaaaaaaaaaaaaaaaaaaattaaagctaaattagtgccatttcctaatttattagtaatttaacaagtcctgcaaactgtgcttgcatgataattaggagtcgcggtcagccccaattccctttttccataggccttgttttatagatagattgatagatagtactagtctgttttcacaccgctataaaaaaaaaatccaagactgggtaatttataacggaaagaggtgtaattgactcaccgttccacatgtctggagaggcctcagaaaactcagtcatggcagaaggggaagaaagaagcttcttctcatggcggcaggagggagaagtatgtgagggtgcaggaaaaacgaacatttataaaaccatgagatctcgtgagaattcactcactatcatgagaacagcggcgggggaaccacccccgtaatccaatcatttccctccctcgacacgtggggactacattttgagatgaaatttgggggggaacacagagtcaaactatatcataaatatatatttaatacattttactatttgtttcttttttgagatggggttattactctgttgccaaggctcacctggaactcctaggttcaggtgcttctgcctcagcctcttgagtatctgtcattataggtgcatcccaccatgcctgacagatgtattatatttagtgcctatttttgcagtttacagatgtttttaggaaaacttacataagaattatagatcaggccaggcatggtggctcacgcctataatccccacactttgggaggccgaggtgggaggatcacttgaggacatgagtttgagaccagcctggccaacatggtgaaaccctatctctactacaaatacaaaaattagctgggggtggtggcaggcacctgtaatcccagcccttcgggaagttgaggtgggagaatcgtttgaaccccagaggcagagattgcagtgagctgagatcgcgccactgcactccagcctgggcgacagagtcagaccctgtctaaaataataataataataattataggtcaggggcagtggctcacacctataatcctagcatcttggaaggctgaggtgggaggatagcttgagctcaggagttcaagaccagcctggacaacatggtgagaccctgtctctataaaagatttttttttttttttgagacagagtcttgctctattgccaggctggagtgcagtggcacagtcttggctcactgcaaccgcctcccgggttcaagtgattctcctgcctcagcctccagagtagctgggactataggcgcctgccaccacacccagctaatttttgtatttttattagacacagggtttcaccatgttggccaggatggtttttttttttttctcttcttgacctcgtgatccacccgccttggcctcccaaagtgctgggattacagacatgagccaccgtgcccggccttttttttttttatttttaattagccatgcgtagtgttgtgtgctcgtagtcccagcctcttgggtggctgaggtgggaggattgcttgagcctaggaggtcaaggctgcagtgagctatgatcaagccactgtcctccaggttgggtgataaagtgagaccttgtctcgagaaaaaaaaaaaaaaagcattgggccaggcgcagtggctcacacctataatttgaccactttgggaagccaagacggatggatcacctgaggtcaggagttcaagacctgcctggccaacatggtgaaaccctgtctatactaaaaaatacaataattaactgggcatggtggtgtgtgcctgtagtcccagctactcgggtggctaaggcacgaaaatcgcttgaacccgggaggtgaaggttgcagtgagccgagattgtgccactgcactacagcttgggtgacagagtgagaccctgtctcgaaagaaaaaaaaaaaagcataaaaatccatcaaaaaattataatacattccttgaactatttctcaaggttgttaaaggtattaaacaagacgtactagatgctctgtacacagggatactaataatgagagaccttatcttaccccccatcaaaatagactaagtccgtacaagatactggcacaagaaaggatcttacaggttgtgacaattaagtagaatgatggaccttggcccgatcctacaggttgtgagagttaagtcaaatggatattgggatgctcaataaattagcatccttatctcccaactatggactttcccgcatcttggagcaagagcaagtaattctcttttctgagcctcttagcgtcccctctgagaaatagggaaatgcacagcctggttgtgggggctcagcgacgctccgcacagcgcctggcacttggtacaggcgccagtagagggcggtgttgaggcgtctattgcgattaagacggtaggcgatgagggaagctaagtgggtctctctcctgccttcccgcagctgagtgcacggttatgggaatccccagtatctccaccaatctctccggcttcggctgcttcatggaggaacacatcgcagacccctcagcttacggtcagcgcccaggcccctgaggattctggaagggggcttgagaacagtgactcctggatcctgggaaggaggagcttgcacccttggagaccaggggcaccaggaaatgggggggctctcgggccgctgggagaggaggggtcctggtgccacagagtcctggagaccatgaagatctccccaggatgttttctgaacctgggtcctggcctccacaggtatctacattcttgaccggcggttccgcagcctggatgattcctgctcgcagctcacctccttcctctacagtttctgtcagcagagccggcggcagcgtatcatccagcggaaccgcacggagcgcctctccgaccttctggactggaaatacctaggccgggtaggacccccactttcctatcctctcctggcaaatcagcagctcctggctaggggtctttggtgcagggtgcagggtctcatcctacttgtaacagttgctgtccctttaagcaatgaaccaggccgatcgcgatggtggctcatggttgtaatcccagcactttgggaggcctaacaggaaggatcgcatgagctcaggagttaagagaccagcctgggcaacattttatttttactaaaaataaaaaaaaatcagccgagcgtgatggtgcgtgcctgtagtcccagctactggggaggctgaggtggtagggtcgcttgagccccggaggttgaggctgcagtgagccttgttcaaacccactgcactccatcctagagtaacagaacaagaccgtgtttaaaaagaaaaaaagaacattggtcaggcgttctcatttgcataagggtgtggccaaagaggagactcacttgagaattcggcccctgtttgaggggtatggctcagaaactgtccattcacaggtctgcttatctacataaggggtggagtcagatagatggattatttgtatggggtgcagcagtgtgctcatctgcataaagggtgtggccaagacccccatcatctgcctgggggtggggcctggctctcacatcctctgccccctctcctcctttcccctagtactatatgtctgcgcgccacatggcgctgtccaaggcctttccagagcacttcacctacgagcccaacgaggcggatgcggtgagtggaccctggatttctgcttagccaggagctaaagggctgggcttcggtggggtgggggtccctcctggagtgggaaggcctagaggtggctggatgcctgggtgggactgtgaggtcctcagctcaccctggtttgcacccacatcgccgtatgcaggcccaggggtaccgctacccacggccagcctcggtgccaccgtcgccctcgctgtcacgacactccagcccgcaccagagtgaggacgaggaggatccccggaacgggccgctggaggaagacggcgagcgctacgatgaggacgaggaggccgccaaggaccggcgcaacatccgtgcaccagagtggccgcgccgagcgtcctgcacctcctccaccagcggcagcaagcgcaactctgtggacacggccacctccagctcactcagcaccccgagcgagcccctcagccccaccagctccctgggcgaggagcgtaactaagtccgccccaccacactccccgcctgtcctgcctctctgctccagagagaggatgcagaggggtgctgctcctaaacccccgctccagatctgcactgggtgtggccccgcagtgcccccacccagtccgccaaacactccaccccctccagctccagtttccaagttcctgcactccagaatccacaaagccgtgcctttctctggctccagaatatgcataatcagcgccctggagtcccctgggcctggaccgcttcccagaggccaggaatctgccattactctgcggtggtgccagaggttttaggaaacctggcatggtgctttcaggtctggggcttttagagccccccgtgtggcttacaaattctacagcatacagagcaggccacgctcaggcccggcatgcgggccaccaagttctggaaaccacgtggtgtccctgcgaatggggcgatcaagtccagagccggggcactttcagagtttgaaggtaactgagagcagatggtcctccatttcaactccagaagtggggctctgggagggatgttctagccctccctggcatgtcagagccaggctctgcctggaggatccctccatccggctcctgtcatcccctacactttggccaagcaagaggtggtagaaccacttggctgctcattccttctggaggacacacagtctcagtccagatgccttcctgtctttctggccctttctggaccagatcctactcttcctttctaaatctgagatctccctccagggaatccgcctgcagaggacagagctggctgtcttcccccacccctaacctggcttattcccaactgctctgcccactgtgaaaccactaggttctaggtcctggcttctagatctggaaccttaccacgttactgcatactgatccctttcccatgatccagaactgaggtcactgggttctagaacccccacatttacctcgaggctcttccatccccaaactgtgccctgccttcagctttggtgaaagggagggcccctcatgtgtgctgtgctgtgtctgcaccgcttggtttgcagttgagaggggagggcaggaggggtgtgattggagtgtgtccggagatgagatgaaaaaaatacatctatatttaagaatcccaggtgtggtcagacatgacacttggtgggcctggctgtgacgtccccccagtaccaccccttccctggtctgagctcatcctcttattctagagagacttggggggtctccctatgttgcttaggctggtctcaacctcctgggctcaagtgatcgtcctgccttggcctcccaaagtgctgggattacaggcttgaaccaccctgccccagacccaggggaaggagaaggggaagagtcagagctgacacagcagttttgcagcttggacacagcaagtgctttttaaggcctgttttaagcttcttcagttcccatttggagaatccggcccaaggtttcacatcccatttctgtgatgaaccctaaaccccttgtccagcattgcttgggcatcacgtgttcctggctgctcagctccactggccagttcctctcagtgtgcgcctcacattgggatcactcaagctcgggccataatctcttaaactctggagcaaagacctcagtgagccccccggttgccatgcaagttgcatagcaatagcgtcaatgtgcccatataaagggcctaataattccaactgcctcccagaattgaggcttttttcttttttgagacagagtcttactctgttgcccaagctggagcacagtggcgccatctcagctcaccgcaacttccgtgaggcagacatgttttttgtttgagatgggagttccactctttttgcccaggctggagtgcaatgacgccatctcagctcactgcaacctccgcctcccagattcaagcgattctcctacctcaacctcctgactagctgggattacaggtatgagccaccacacccagctaattttgtattttagtagagacggggtttcttcatgttggtcaggctggtctcgaactcctgacatcaggtgagcgacccacctggccccataatcactcgttaaatagggctgtttctggctgagacctaacatctgtttctagataccaacctaggtcttaattcaactccctggagtctatctctaccatctgggagggacagttaacagccaggcaagcagagaaccctcaggttcaacactacccagatagcctcttgtcttaggacagttaagaaaatgggagatggggccaactcctctttcactggcatcccaacccagccagaagctttatgagaacagatcccacctcatcttccacaccacacacaaaaccaccagctgcatcaaaaagctttatttccatttggtccaaggcttgttaggatagttaagaaagctgcctattggctggagggagaggcttaggcagaagccctattactttgcaaggggcccttcagaagtcgctgggctcagaaggctcttagtcgtgcttgagagtgagcctttcgaagagatactcgcccagcccagcctccgggccacccagcctgtggaggttggtcaggtggtcacccatcttcttgataagcttcacttcctcatctaggaagtgagtctccaggaagtcacagagctgagagagaagagggagaaattagaaacccaatccctggaaaagagggggagagagcagccagttgcagattaaaatgtgacaggtgtgaaatgaggctctgaaggaaatttgcccaaagggagcagaggcttgaggggtatggttgggtagccatggatgcagcgggtacgtacatgggggtccgtgcgggcagaacccagggcatgaagatccaaaagggcctggttcagctttttctccagggccatggcagctttcatggcgtctggggttttaccccactcatcttcagctggcttctatatagaagggagaaatggctcagaatacacacactcggcacatagaactaaacctacatttcccaagagtcgttgggggtcagaggcccaggccacaaagacatgtgacagcttgtatttatcactctccgcaccctgtcctagctcttacag